(19) United States
(12) Patent Application Publication
(10) Pub. No.: US 2024/0000359 A1
(43) Pub. Date: Jan. 4, 2024

(54) MAGNETOENCEPHALOGRAPHY METHOD AND SYSTEM

(71) Applicant: The University of Nottingham, Nottingham, Nottinghamshire (GB)

(72) Inventors: Matthew BROOKES, Nottingham, Nottinghamshire (GB); Elena Boto, Nottingham, Nottinghamshire (GB)

(21) Appl. No.: 18/247,313

(22) PCT Filed: Sep. 30, 2021
(86) PCT No.: PCT/GB2021/052537

§ 371 Date: Mar. 30, 2023

(30) Foreign Application Priority Data
Sep. 30, 2020 (GB) 2015427.4

(51) Int. Cl. A61B 5/245; G01R 33/00; G01R 33/26; G01R 33/02; G01R 33/032

(52) U.S. Cl. CPC A61B 5/245; G01R 33/0094; G01R 33/26; G01R 33/0206; G01R 33/032

(57) ABSTRACT

A method of reducing error in magnetoencephalography arising from the presence of a non-neuromagnetic field. The method comprises measuring, using a sensor array for measuring neuromagnetic fields, magnetic field at a plurality of discrete locations around a subject's head to provide sensor data; wherein the magnetic field measured at at least some of the locations includes a neuromagnetic field from a source of interest within a subject's brain and a non-neuromagnetic field from a source of no interest external to the brain. The measuring comprises: measuring, at at least a first subset of the locations, a magnetic field along a first direction relative to a radial axis intersecting the respective location, and measuring, at at least a second subset of the locations, a magnetic field along a second direction relative to a radial axis intersecting the respective location which is different to the first direction; and performing source reconstruction using the sensor data.

### TECHNICAL FIELD

This invention relates generally to magnetoencephalography (MEG), particularly to methods of reducing errors in MEG associated with non-neuromagnetic fields.

### BACKGROUND TO THE INVENTION

Magnetoencephalography (MEG) is a non-invasive functional neuroimaging technique involving the measurement of magnetic fields generated by current flow through neuronal assembles in the brain (known as neuromagnetic fields) at discrete locations around the scalp. Mathematical modelling based on the neuromagnetic field measurements (termed source reconstruction) enables generation of three dimensional (3D) images showing moment-to-moment changes in neuronal current flow. In this way, MEG offers a unique non-invasive imaging technique for studying brain function, enabling one to track activity within (and connectivity between) brain regions in real time, as those regions become engaged to support cognition. MEG is therefore a powerful tool for basic neuroscience and a useful clinical metric, particularly in disorders like epilepsy which involve abhorrent electrophysiology.

The magnetic fields generated by brain activity are extremely small, typically of order 100 fT, and requires an array of highly sensitive magnetometers to be measured. Until recently the only magnetometer with sufficient sensitivity for their measurement was the superconducting quantum interference device (SQUID). SQUIDs provide sensitivities of approximately 2-10 fT/√Hz, but require cooling to cryogenic temperatures to operate which brings with it a number of practical and functional draw-backs that have limited the utility of MEG. Recent years have seen the development of new generations of high sensitivity magnetometers that do not require cryogenic cooling. One such sensing technology that is fundamentally changing the MEG field is the optically pumped magnetometer (OPM) which offers field sensitivity similar to that of a SQUID (noise levels of approximately 7-15 fT/√Hz) without cryogenic cooling. Device miniaturisation means that OPMs can be made small and lightweight, offering new sensor mounting configurations such as lightweight wearable helmet designs that are not possible with SQUIDs, and making them ideal for functional neuroimaging (see R. M. Hill et al. “Multi-channel whole-head OPM-MEG: Helmet design and a comparison with a conventional system” Neuroimage 219, 116995, 2020).

Despite the advances in sensing technology, one of the key challenges that remains to be adequately addressed is to minimise interference/error from non-neuromagnetic fields on MEG measurements. Because the neuromagnetic fields being measured are so small, any background magnetic field, such as the earth's magnetic field or fields caused by nearby electrical equipment or magnetic objects, can degrade or introduce errors and/or artifacts to the MEG measurements and resulting source reconstruction. MEG is particularly susceptible to errors from the presence of dynamic background fields and movements of the head/sensors in a static field that translates to a dynamic field seen by the sensors.

Traditional approaches to reducing these errors include minimising the background fields using shielding techniques, and compensating for the background field using additional reference field measurements or complex signal processing techniques such as signal space projection and signal space separation. For example, a MEG system is typically housed within a magnetically shielded room (MSR), and all other external sources of magnetic field within the MSR are removed or minimised as much as possible. Active field nulling coils have also been proposed to control/minimise background magnetic field in the vicinity of the sensors (see, e.g., E. Boto et al. “Moving magnetoencephalography towards real-world applications with a wearable helmet” Nature 555, 657, 2018). Using these shielding methods, background fields can be as low as 1 nT. However, even in these very low field environments the associated errors and artifacts in the MEG measurements can be of a similar magnitude to the measured brain activity of interest (˜100 fT). Axial or planar gradiometer configurations have been successfully applied and are well suited to SQUID-MEG systems. For example, in an axial SQUID gradiometer, two coils are wound in series, with one positioned further from the scalp (typically by 5 cm) to obtain reference measurements of the background field (and noise) that can be subtracted/removed from the field measurements obtained from the coil positioned closer to the scalp to isolate the signal of interest. However, such solutions add to the complexity, bulk and weight of the sensor array, and are therefore not suitable or practical for wearable helmet configurations that take advantage of lightweight sensing technologies such as OPMs.

Alternative ways to reduce or suppress errors associated with background non-neuromagnetic fields are therefore needed for the further advancement of the MEG field. Aspects and embodiments of the present invention have been devised with the foregoing problem in mind.

### Statements of Invention

According to a first aspect of the invention, there is provided a method of reducing error in magnetoencephalography (MEG) associated with the presence of, or arising from the presence of, a non-neuromagnetic field. The method comprises measuring, using a sensor array for measuring neuromagnetic fields, magnetic field at a plurality of discrete locations around a subject's head to provide sensor data. Each discrete location may be associated with a sensor, and vice versa. The magnetic field measured at some or all of the locations may include a (contribution from a) neuromagnetic field from a source of interest within a subject's brain and a (contribution from a) non-neuromagnetic field from a source of no interest external to the brain. Measuring the magnetic fields may comprise measuring, at at least a first subset of the locations, or at some or all of the locations, a magnetic field component along a first direction relative to a radial axis intersecting the respective location. The radial axis may be defined with respect to the subject's head, a sphere approximating the subject's head, or the local curvature of the head, at the respective location. For example, the radial axis may be perpendicular to the tangent of the local curvature of the head at the respective location.

Measuring the magnetic field may further comprise measuring, at at least a second subset of the locations, or at some or all of the locations, a magnetic field component along a second direction relative to a radial axis intersecting the respective location which is different to the first direction. The sensor data may comprise at least one magnetic field component measured at each location. The sensor data may comprise a plurality of measurement channels, at least one channel for each location, where each channel contains a magnetic field measurement for a given direction at a given location. The method may further comprise performing source reconstruction using the sensor data. A source of interest may be associated with a localised current flow, such as a current dipole, characterised by a timecourse (a time varying signal), orientation, and/or a location in the brain. Source reconstruction may comprise determining or deriving a timecourse, orientation, and/or a location of the source of interest from the sensor data. Source reconstruction may comprise determining or deriving an image of neuronal activity within the brain from the sensor data.

The non-neuromagnetic field may be a background magnetic field that interferes with the measurement of neuromagnetic fields from the source of interest and introduces error in MEG, such as error in the reconstructed timecourse, orientation and/or location of the source of interest.

Prior art methods of reducing errors associated with non-neuromagnetic fields involve suppressing/cancelling the background fields themselves (e.g. using magnetic screening/shielding), or compensating for the background field using gradiometer or reference array configurations or advanced signal processing techniques that try to extract the signal of interest in the MEG measurements. Shielding methods do not fully eliminate the background field, gradiometer/reference array solutions are bulky, cumbersome and limit the utility of MEG (and still do not fully eliminate the background field), and signal processing techniques are computationally complex and expensive. By contrast, the present method provides a solution to this problem based on manipulating the magnetic field information obtained at the sensor space level, and leveraging the noise reduction provided by source reconstruction/localisation techniques to minimise the interference of the non-neuro magnetic fields. This provides a simple and effective way to improve the robustness of MEG to background non-neuromagnetic fields and movements therein without using bulky sensor arrays and complex signal analysis techniques. It may also permit the use of less shielding.

More specifically, the method is based on new technical insights that measuring magnetic field in different directions/orientations across the plurality of locations alters the information obtained from the sensor array about the measured magnetic field topography or field pattern from, or associated with, a source of no interest external to the brain, which in turn reduces the correlation with the magnetic field topography or field pattern from, or associated with, a source of interest within the brain. Once source reconstruction/localisation is applied to the measured sensor data, the reduced correlation reduces or suppresses error associated with the presence of non-neuromagnetic fields, such as error in the reconstructed timecourse, orientation and/or location of the source of interest.

MEG error may be defined by a deviation in the reconstructed timecourse, orientation and/or location of the source of interest from the true timecourse, orientation and/or location. The measured magnetic field topography or field pattern from an internal source of interest and an external source of no interest may be described by a respective field vector, which may contain the location and orientation of the sensors and the field measurements. The correlation may be characterised by a correlation parameter, such as the Pearson correlation coefficient for the respective field vectors.

The non-neuromagnetic field may be or include a substantially spatially uniform background magnetic field, such as that from the earth's magnetic field. Alternatively or additionally, the non-neuromagnetic field may be or include a spatially non-uniform background magnetic field, such as that generated from a source of magnetic field in proximity to the sensor array, e.g. other biomagnetic fields generated by the subject's body (such as the heart) and electrical equipment. The non-neuromagnetic field may be or include a static background magnetic field and/or a dynamic background magnetic field. A dynamic background magnetic field may be generated from a source of magnetic field in proximity to the sensor array, e.g. other biomagnetic fields generated by the subject's body (such as the heart) and electrical equipment. Alternatively, a dynamic background magnetic field may result from relative movement between the sensor array (and head) and a static non-neuromagnetic field, such as when a subject's head moves during the measurement step.

As such, the method may advantageously suppress or reduce motion related artifacts/errors in the reconstructed timecourse, orientation and/or location of the source or interest, improving motion robustness and allowing a subject to move during data acquisition.

The step of source reconstruction may be preceded by a signal processing step, e.g. to remove noise and/or signal artifacts in the sensor data prior to source reconstruction. The signal processing step may include performing any one or more of: signal space separation, signal space projection, independent component analysis, and principle component analysis. Such processing techniques may also benefit from the measurement of different field components across the array and further reduce errors in source reconstruction.

The first direction and the second direction may be substantially orthogonal, or not orthogonal. Substantially orthogonal may mean within +/−5 degrees of orthogonal. Where they are not orthogonal, the first and second directions may make an angle between substantially 20 and 90 degrees (e.g. between 30 and 90, 40 and 90, 50 and 90, 60 and 90, 70 and 90, or 80 and 90 degrees, or any combination or subrange thereof). The first direction and the second direction may be substantially the same at each location.

The method may comprise measuring, at each location or at least some locations, a magnetic field (component) along both the first direction and the second direction. Measuring multiple different magnetic fields (components) at the same location increases the number of measurement channels in the sensor data and the total field measured from the source of interest, which in turn reduces the error in the reconstructed timecourse, orientation and/or location of the source of interest. This also contributes to reducing the correlation between the measured field pattern from the source of interest and the non-neuromagnetic field which in turn further reduces the error in the reconstructed timecourse, orientation and/or location of the source of interest

The plurality of locations may consist of the first and second subsets, or it may include additional subsets of sensors (e.g. measuring field in different directions/orientations).

The method may further comprise measuring, at at least a third subset of the locations, a magnetic field (component) along a third direction relative to a radial axis intersecting the respective location which is different to the first direction and the second direction. The third direction may be substantially orthogonal to the first direction and/or the second direction. Alternatively, the third direction may not be orthogonal to the first direction and/or the second direction. Where the third direction is not orthogonal to the first direction and/or the second direction, it may make an angle with the first and/or second direction between substantially 20 and 90 degrees (e.g. between 30 and 90, 40 and 90, 50 and 90, 60 and 90, 70 and 90, or 80 and 90 degrees, or any combination or subrange thereof). The third direction may be substantially the same at each location.

The method may comprise measuring, at each location or at least some locations, a magnetic field along each of the first, second and third directions. In this case, the method may comprise measuring, at each location or at least some locations, the (3D) magnetic field vector.

In an embodiment, the second direction is a substantially radial direction or is aligned substantially/approximately parallel to a radial axis at the respective location. In this case, the first and/or third direction may be a tangential direction, or aligned substantially/approximately parallel to a tangential axis at the respective location (i.e. substantially/approximately tangential to the scalp/head's surface at the respective location). For example, where field is measured in the first and second direction, the first direction may vary, such that the first direction for each sensor in the first subset lies in the tangential plane at the respective location.

Source reconstruction may be performing using various techniques known in the art, such as a beamformer, a dipole fit approach, a minimum-norm estimate approach, or a machine learning approach. In principle, all source reconstruction techniques will benefit from the reduced correlation between the neuromagnetic field and non-neuromagnetic field contributions in the sensor data provided by the present method.

In an embodiment, source reconstruction comprises using a beamformer approach. A beamformer has a non-linear dependence of the error on the correlation parameter meaning that even small reductions in correlation can have a significant impact on the resulting source reconstruction error.

Source reconstruction may be performed using a processing module. The processing module may comprise one or more processors. The processing module may be configured to receive the sensor data from the sensor array.

In an embodiment, magnetic field is measured in a single direction at each location and the sensors are single axis sensors. In another embodiment, where magnetic field is measured in different directions at the same location, the respective sensor may be or comprise a multi-axial magnetometer, e.g. a bi/dual axis or triaxial magnetometer.

In an embodiment, each sensor is or comprises an optically pumped magnetometer (OPM). The OPMs may be mounted on or to a wearable helmet. OPMs advantageously do not require cryogenic cooling to operate, are lightweight and provide flexibility in their placement and orientation in the sensor array/helmet, compared to e.g. SQUID magnetometers that require cryogenic cooling and are fixed in their position and orientation within a cryogenic vessel. The wearable helmet may allow a subject to move during a MEG measurement, the sensors to be closer to the subject's head, and the sensor array to substantially conform to the subject's head, providing higher signal to noise, spatial resolution and measurement uniformity than a SQUID array.

According to a second aspect of the present invention, there is provided the use of a sensor array for measuring neuromagnetic fields at a plurality of discrete locations around a subject's head for reducing error in magnetoencephalography associated with non-neuromagnetic fields. The sensor array may comprise at least a first subset of sensors that are configured to measure a magnetic field along a first direction relative to a radial axis intersecting the respective sensor location; and at least a second subset of sensors that are configured to measure a magnetic field along a second direction relative to a radial axis intersecting the respective sensor location that is different to the first direction.

The error associated with the non-neuromagnetic field may include an error in a reconstructed timecourse, orientation and/or location of a source of interest within the subject's brain. The non-neuromagnetic field may be or include a substantially spatially uniform background magnetic field and/or a spatially non-uniform background magnetic field. The non-neuromagnetic field may be or include a substantially static background magnetic field and/or a dynamic background magnetic field. The dynamic background magnetic field may result from relative movement of the sensor array and the non-neuromagnetic field, e.g. movement of the sensor array in the background non-neuromagnetic field.

The sensor array may comprise at least a third subset of sensors that are configured to measure a magnetic field along a third direction relative to a radial axis intersecting the respective sensor location which is different to the first direction and the second direction.

The sensor array may comprise at least 20, 25, 30, 35, or 40 sensors. The first and/or third subset of sensors may include at least 2, 3, 4 or 5 sensors.

All of the sensors or at least some of the sensors may be single-axis sensors configured to measure a magnetic field along a single direction. In this case, the field sensitive axis of the sensor may be oriented or rotated to measure field in any given direction.

All of the sensors or at least some of the sensors may be bi-axial sensors configured to measure a magnetic field along two different directions including the first direction and the second/third direction.

All of the sensors or at least some of the sensors may be tri-axial sensors configured to measure a magnetic field along three different directions including the first, second and third directions.

The first direction and the second direction may be substantially orthogonal, or not orthogonal. Where they are not orthogonal, they may make an angle between substantially 20 and 90 degrees (e.g. between 30 and 90, 40 and 90, 50 and 90, 60 and 90, 70 and 90, or 80 and 90 degrees, or any combination or subrange thereof). The first direction and the second direction may be substantially the same at each sensor location.

The third direction may be substantially orthogonal to the first direction and/or the second direction. Alternatively, the third direction may not be orthogonal to the first direction and/or the second direction. Where it is not orthogonal to the first direction and/or the second direction, the third direction may make an angle with first direction and/or the second direction of between substantially 20 and 85 degrees. The third direction may be substantially the same at each sensor location.

In an embodiment, the second direction is aligned substantially parallel to the radial axis at the respective sensor location.

Each sensor may be or comprise an optically pumped magnetometer (OPM). The OPMs may be mounted on or to a wearable helmet. In an embodiment, the sensors are triaxial OPMs and the first, second and third directions are substantially orthogonal.

The advantages described for the first aspect apply equally to the second aspect.

According to a third aspect of the invention, there is provided a magnetoencephalography (MEG) system. The system may be configured to perform the method of the first aspect. The system may comprise a sensor array for measuring neuromagnetic fields at a plurality of discrete locations around a subject's head and output or provide/generate sensor data. The sensor array may comprise a plurality of sensors. At least a first subset of sensors may be configured to measure a magnetic field along a first direction relative to a radial axis intersecting the respective sensor location. At least a second subset of sensors may be configured to measure a magnetic field along a second direction relative to a radial axis intersecting the respective sensor location that is different to the first direction. The system may further comprise a processing module configured to perform source reconstruction using the sensor data. The sensor data may comprise at least one magnetic field measured at each sensor location. At least some of the measured magnetic fields may include a neuromagnetic field from a source of interest within a subject's brain and a non-neuromagnetic field from a source of no interest external to the brain. The system may be configured to reduce error in magnetoencephalography associated with the non-neuromagnetic field. The sensor data may comprise a plurality of measurement channels, at least one channel for each location, where each channel contains a magnetic field measurement for a given direction at a given location. Source reconstruction may comprise determining or deriving a timecourse, orientation and/or a location of the source of interest from the sensor data. Source reconstruction may comprise determining or deriving an image of neuronal activity within the brain from the sensor data. The processing module may comprise one or more processors and be configured to receive the sensor data from the sensor array.

The error associated with the non-neuromagnetic field may include an error in a reconstructed timecourse, orientation and/or a location of the source of interest within the subject's brain. The non-neuromagnetic field may include a substantially spatially uniform background magnetic field and/or a spatially non-uniform background magnetic field. The non-neuromagnetic field may include a substantially static background magnetic field and/or a dynamic background magnetic field. The dynamic background magnetic field may result from relative movement between the sensor array and the non-neuromagnetic field, e.g. movement of the sensor array in the background non-neuromagnetic field.

The sensor array may comprise at least a third subset of sensors that are configured to measure a magnetic field along a third direction relative to a radial axis intersecting the respective sensor location which is different to the first direction and the second direction.

The sensor array may comprise at least 20, 25, 30, 35 or 40 sensors. The first and/or third subset of sensors may include at least 2, 3, 4, or 5 sensors.

Each sensor of the array may be or comprise an optically pumped magnetometer. The system may further comprise a wearable helmet comprising the sensor array. The helmet may be substantially rigid or flexible. The system may be configured to reduce error in a reconstructed timecourse and/or a location of the source of interest within the subject's brain resulting from relative movement between the helmet and the non-neuromagnetic field.

All of the sensors or at least some of the sensors may be single-axis sensors configured to measure a magnetic field along a single direction. Tn this case, different field directions/components are measured by orienting/rotating the field sensitive axis of the sensor.

All of the sensors or at least some of the sensors may be bi-axial sensors configured to measure a magnetic field along two different directions including the first direction and the second/third direction.

All of the sensors or at least some of the sensors may be tri-axial sensors configured to measure a magnetic field along three different direction including the first, second and third directions.

The first direction and the second direction may be substantially orthogonal, or not orthogonal. Where they are not orthogonal, they may make an angle between substantially 20 and 90 degrees (e.g. between and 90, 40 and 90, 50 and 90, 60 and 90, 70 and 90, or 80 and 90 degrees, or any combination or subrange thereof). The first direction and the second direction may be substantially the same at each sensor location.

The third direction may be substantially orthogonal to the first direction and/or the second direction. Alternatively, the third direction may not be orthogonal to the first direction and/or the second direction. Where it is not orthogonal to the first direction and/or the second direction, the third direction may make an angle with first direction and/or the second direction of between substantially 20 and 90 degrees (e.g. between 30 and 90, 40 and 90, 50 and 90, 60 and 90, 70 and 90, or 80 and 90 degrees, or any combination or subrange thereof). The third direction may be substantially the same at each sensor location.

In an embodiment, the sensors are triaxial OPMs and the first, second and third directions are substantially orthogonal.

In an embodiment, the second direction is aligned substantially parallel to the radial axis at the respective sensor location.

The processing module may be configured to perform source reconstruction using a beamformer.

The system may further comprise a room or enclosure configured to suppress background magnetic field at least at the location of the sensor array, optionally to between 0.1 nT and 10 nT, and optionally in the frequency range 0 Hz to 500 Hz. The room or enclosure may comprise metal shielded walls and/or one or more electromagnetic field nulling coils.

The system may further comprising one of more pieces of electrical equipment, such as measurement and/or stimulus equipment, at least partially located within the room or enclosure. Optionally, the stimulus equipment may include an electronic display device and/or head mounted display device.

Advantages described for the first aspect apply equally to the third aspect.

Features which are described in the context of separate aspects and embodiments of the invention may be used together and/or be interchangeable. Similarly, where features are, for brevity, described in the context of a single embodiment, these may also be provided separately or in any suitable sub-combination. Features described in connection with the method can have corresponding features definable with respect to the use and system, and vice versa, and these embodiments are specifically envisaged.

### BRIEF DESCRIPTION OF DRAWINGS

In order that the invention can be well understood, embodiments will now be discussed by way of example only with reference to the accompanying drawings, in which:

FIG. 1 shows a schematic magnetoencephalography (MEG) system according to an embodiment;

FIG. 2 shows a method of reducing error in MEG according to an embodiment;

FIGS. 3a and 3b show schematic diagrams of a neuromagnetic and non-neuromagnetic field respectively;

FIGS. 4a to 4c show the location and orientation of sensors in three different sensor array configurations simulated;

FIGS. 5a to 5c show perspective, side and front views of an example vector magnetic field from a source of interest detected by the sensor array in FIG. 4a;

FIGS. 6a to 6c show field maps of the radial, polar and azimuth magnetic field components at each sensor location for the field vector in FIGS. 5a-5c;

FIG. 6d shows a field map of the radial magnetic field component at each sensor location for the same source as in FIGS. 5a-5c but for the sensor array in FIG. 4c;

FIGS. 7a and 7b show histograms of the calculated frobenius norm (∥l∥) values of the forward field, and the mean ∥l∥ values when the source location is varied for the field components shown in FIGS. 6a-d, respectively;

FIG. 7c shows the total beamformer error against ∥l∥ for source location and each array in FIG. 4a-4c;

FIG. 8 shows the dependence of ∥l∥ on number of channels (sensors) for the array configuration shown in FIG. 4a compared to the fixed values for the arrays shown in FIGS. 4b and 4c (horizontal lines);

FIG. 9 shows the dependence of various parameters from equations 17 and 18 on the error from an external source of non-neuromagnetic field (left column), sensor noise (centre column) and total beamformer error (right column);

FIG. 10a shows the mean correlation parameter for internal (left graph) and external (right graph) sources for each array of FIG. 4a-4c;

FIG. 10b shows an example vector magnetic field at each sensor of the array of FIG. 4c from an internal and an external source;

FIG. 10c shows field maps of the radial, polar and azimuth field components at each sensor location in FIG. 10b;

FIG. 11a shows example beamformer images and reconstructed timecourses for the arrays of FIGS. 4a-4c for no external source interference (left column) and including external source interference (right column) for each array of FIGS. 4a-4c;

FIGS. 11b to 11d show the corresponding timecourse correlation, timecourse error and localisation error for each array in FIGS. 4a-4c as a function of external interference amplitude;

FIGS. 12a-12c shows corresponding timecourse correlation, timecourse error and localisation accuracy for each array of FIGS. 4a-4c as a function of internal interference amplitude;

FIG. 13a shows the effect of motion in a static field on the field measured at each sensor and a resulting motion artefact in the array of FIG. 4a;

FIG. 13b shows the calculated timecourse correlation, timecourse reconstruction error, and localisation error resulting from motion for each of the arrays in FIGS. 4a-4c;

FIG. 14a shows the location and orientation of sensors for a simulated 50-sensor radial array (left) and a “mixed” array where five sensors have been arranged to measure tangential field;

FIG. 14b shows the resulting measured field distribution for an internal and external source for the arrays shown in FIG. 14a;

FIG. 14c shows the calculated correlation parameter for the two source distributions in FIG. 14b for different source positions;

FIGS. 14d to 14f show the corresponding timecourse correlation, timecourse error and localisation error for the arrays in FIGS. 14a and 14b as a function of external interference amplitude;

FIG. 15a shows the location and orientation of sensors in an experimental 45-sensor radial array (left) and a “mixed” array where five sensors have been arranged to measure tangential field;

FIG. 15b shows amplitude spectra of sensor data from each array in FIG. 15a, the inset shows close up of an interference signal, and the distribution of the amplitude of the interference signal is shown on the right for each array in FIG. 15a;

FIG. 15c shows a beamformer output image for the timecourse overlaid on a model of a brain and a reconstructed timecourse (right hand line plots) for each array in FIG. 15a;

FIG. 15d shows the calculated correlation parameter for the signal of interest and the interference signal shown in FIG. 15b at each region of the brain for both arrays shown in FIG. 15a;

FIG. 15e shows amplitude spectra of reconstructed timecourse from each array in FIG. 15a, the inset shows close up of the interference signal, and the difference in the interference signal amplitude for each region of the brain is shown in on the right hand image;

FIG. 16 shows the source reconstruction error plotted against the correlation parameter for a dipole fit and a beamformer with varying sensor noise; and

FIGS. 17a, 17e and 17i show magnetic resonance images (MRIs) of an adult, 4-year old child and a 2-year old child, respectively;

FIGS. 17b, 17f, and 17j show a three dimensional rendering of the head geometry for an adult, a 4-year-old, and a 2-year-old, based on the MRIs of FIGS. 17a, 17e and 17i;

FIGS. 17c, 17g, and 17k show simulations of the sensitivity of a radially oriented sensor array to dipole source location within the brain; and

FIGS. 17d, 17h, and 17i show simulations of the sensitivity of a triaxial sensor array to dipole source location within the brain.

It should be noted that the figures are diagrammatic and may not be drawn to scale. Relative dimensions and proportions of parts of these figures may have been shown exaggerated or reduced in size, for the sake of clarity and convenience in the drawings. The same reference signs are generally used to refer to corresponding or similar features in modified and/or different embodiments.

### DETAILED DESCRIPTION

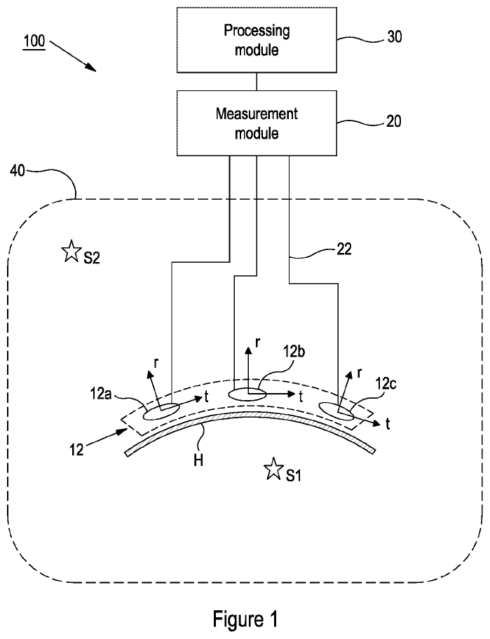

FIG. 1 shows a schematic magnetoencephalography (MEG) system 100 according to an embodiment of the invention. The system 100 comprises an array 12 of magnetometers (referred to hereafter as sensors) 12a-12c configured to measure neuromagnetic fields at a plurality of discrete locations around a subject's head H and provide or output sensor measurement data to a measurement apparatus 20. Each sensor 12a-12c is associated with a different discrete location. Although only three sensors 12a-12c are shown, it will be appreciated that in practice a larger number, e.g. 20, 30, 40, 50 or greater, will be included. In an embodiment, the sensor array comprises 50 sensors 12a-12c.

The sensors 12a-12c must be sensitive enough to detect neuromagnetic fields as small as 100 fT. In practice, this means the sensors 12a-12c should have a sensitivity or noise equivalent field of less than 20 fT/√Hz depending on the sensor type and frequency of operation. In an embodiment, the sensors 12a-12c are optically pumped magnetometers (OPMs) mountable on/to a wearable helmet (not shown) configured to fit the subject's head H. Each OPM 12a-12c is a self-contained unit containing a gas vapour cell of alkali atoms, a laser for optical pumping, and on-board electromagnetic coils for nulling the background field within the cell, as is known in the art. The basic operation principle is that optical pumping aligns the spins of the alkali atoms giving the vapour a bulk magnetic property which can be altered by the presence of an external magnetic field and measured by monitoring how the transmission of the laser beam is modulated by the vapour cell.

The MEG measurements are performed in a room or enclosure 40 configured to suppress, attenuate or exclude background magnetic fields within the room using passive and/or active shielding techniques known in the art. For example, the magnetically shielded room (MSR) 40 may comprise a plurality of metal layers, such as copper, aluminium and/or high permeability metal, and one or more electromagnetic (degaussing) coils. The MSR 40 surrounds the subject and the sensor array 12. In an embodiment, the MSR 40 is configured to suppress static background magnetic field to less than 50 nT, preferably less than 10 nT, in order for the OPMs 12a-12c to operate.

The measurement apparatus 20 is located outside the MSR 40 and is connected to the sensor array 12 via shielded leads 22 to minimise electromagnetic interference with the sensor measurements. The measurement apparatus 20 is configured to output one or more signals to the sensor array 12 to operate the sensors 12a-12c and receive or measure one or more signals from the sensor array 12 including sensor measurement data. The one or more output signals may comprise electrical and/or optical signals for data and/or power transmission. The measurement apparatus 20 may comprise a data acquisition module (not shown) with an analogue to digital converter and a memory for receiving and storing the digitised sensor data. Each magnetic field measurement provides a measurement channel. Sensor data comprises a vector of magnetic field measurements, at least one magnetic field measurement or channel for each sensor location.

Sensor data are processed by a processing module 30 to perform source reconstruction/localisation. The processing module 30 may be part of the measurement apparatus 40. Alternatively, the measurement apparatus 20 may be configured to acquire and store the sensor data, and source reconstruction may take place on a separate computing device with the processing module 30. Source reconstruction is a mathematical technique for estimating or reconstructing the location, orientation and time and/or frequency-dependent magnetic signal (timecourse) associated with neuronal activity (current) of the source(s) of interest S1 within the brain based on sensor measurements. It is known as the “inverse problem” and essentially projects the measured fields back into the head and in most cases uses a weighted sum of sensor measurements and a mathematical model of source(s) to predict the current sources. In this way, an image of the neuronal activity within the brain can be generated from the sensor data. In an embodiment, the processing module is configured to perform source reconstruction using a beamformer spatial filter technique, as is known in the art and is discussed in more detail below.

The general method of performing MEG involves measuring magnetic field at a plurality of discrete locations around a subject's head to provide sensor data, and performing source reconstruction using the sensor data. However, in practice, the sensor data includes a neuromagnetic field from one or more sources of interest S1 within a subject's brain and almost always contains artifacts from the presence of a non-neuromagnetic background fields from a source of no interest S2 external to the brain. This leads to errors in source reconstruction. There are three main problems associated with background fields in MEG:

(1) Static field: Even inside the MSR 40, static fields, such as the earth's field, are present albeit substantially attenuated. Static field is not a problem so long as it is low enough for the sensors to operate. For example, OPMs only operate in near-zero field and include on-board field nulling coils to zero the background field in the active sensing region, but these only work up to fields of about 50 nT. If the background field is higher, OPMs simply don't work. Shielding provided by the MSR 40 and electrostatic coils is typically sufficient to reduce static fields to an acceptable level for OPMs.

(2) Static field and movement: A significant advantage of the OPM sensor array 12 over a traditional SQUID array is that it can be integrated into a wearable helmet (not shown), allowing subjects to move during data acquisition. This makes the MEG environment better tolerated by many subjects, but any motion of the head H or sensor array 12 in a background field turns the static field into a dynamic (changing in time) field in the reference frame of the sensors 12a-12c which is measured. This introduces motion artifacts to the MEG measurements that can be larger than brain activity of interest.

(3) Dynamic fields: Whether or not the head H or sensor array 12 moves, there will inevitably be some temporally changing magnetic fields inside the MSR 40, e.g. caused by nearby electrical equipment, large metal objects (cars, lifts etc.) moving nearby, other biological fields generated by the human body (such as the heart), as well as any stimulus equipment. The scale of these fields vary but can be upwards of 100 fT and, e.g. in the case of 50 Hz mains frequency noise, much larger.

As such, the (inevitable) presence of non-neuromagnetic fields can introduce significant error and artifacts to the reconstructed timecourse, orientation and location of a source(s) of interest S1, which should be minimised in MEG.

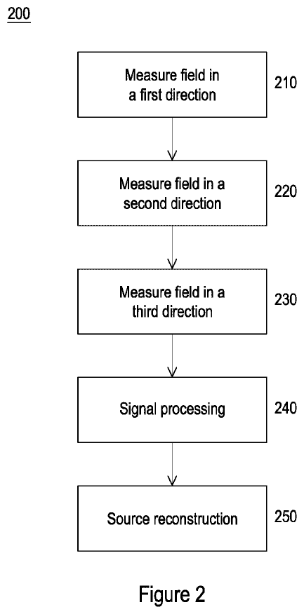

FIG. 2 shows a method 200 of reducing error in MEG associated with non-neuromagnetic fields according to an embodiment of the invention. The method 200 is performed using the MEG system 100. In step 210, magnetic field is measured, at at least a first subset of the locations, along a first direction relative to a radial axis r intersecting the respective location. In step 220, magnetic field is measured, at at least a second subset of the locations, along a second direction relative to a radial axis r intersecting the respective sensor location which is different to the first direction. Optionally, in step 230, magnetic field is measured, at at least a third subset of the locations, along a third direction relative to a radial axis r intersecting the respective sensor location which is different to the first and second directions. In step 250, source reconstruction is performed using the sensor data. Steps S1-S3 may be carried out simultaneously. Step 250 may be preceded by a signal processing step 240 for reducing/removing noise, background field and/or signal artifacts in the sensor data prior to source reconstruction, as is known in the art. For example, step 240 may include performing any one or more of: signal space separation, signal space projection, independent component analysis, and principle component analysis. Such signal processing techniques may also benefit from the measurement of different field components across the array 12 and help to further reduce errors in source reconstruction.

Accordingly, the sensor array of the MEG system 100 comprises at least a first subset of sensors 12a-12c configured to measure a magnetic field along a first direction relative to a radial axis r intersecting the respective sensor location, at least a second subset of sensors 12a-12c configured to measure a magnetic field along a second direction relative to a radial axis r intersecting the respective sensor location that is different to the second direction, and optionally at least a third subset of sensors 12a-12c configured to measure a magnetic field along a second direction relative to a radial axis r intersecting the respective sensor location that is different to the first and second directions. Each sensor 12a-12c can be configured to measure field along a given direction by arranging, rotating or orienting its sensitive axis to along to the desired direction.

In an embodiment, the first and second (and optionally third) directions are substantially orthogonal (i.e. within +1-5 degrees from orthogonal), and the second direction is aligned to the radial axis r. In this case, the second subset of sensors 12a-12c is configured to measure radial components of field, and the first (and optionally third) subset of sensors 12a-12c is configured to measure tangential components of field, i.e. parallel to a tangential axis t with respect to the local curvature at the respective sensor location (see FIG. 1). The tangential axes may include the polar and azimuthal axes. The first direction may be the same for each sensor in the first subset, e.g. the polar or azimuthal axis. Alternatively, the first direction may vary, such that the first direction for each sensor in the first subset lies in the tangential plane at the respective location.

In an embodiment, the sensor array comprises at least 50 sensors 12a-12c and the first subset (and optionally including the third subset) comprises at least 5 sensors. In an embodiment, all the sensors 12a-12c of the array 12 are single axis sensors, i.e. configured to measure magnetic field along a single axis. In another embodiment, all or at least some of the sensors are bi- or dual-axis sensors configured to measure magnetic field along two orthogonal axes. In this case, two field components (e.g. in the first and second directions) can be measured at each location, increasing the number of measurement channels in the sensor data to up to 2N for an N-sensor array. In yet another embodiment, all or at least some of the sensors are tri-axis (or triaxial) sensors configured to measure magnetic field along three orthogonal axes. In this case, three field components (in the first, second and third directions) can be measured at each location, increasing the number of measurement channels in the sensor data to up to 3N for an N-sensor array.

The OPMs' vapour cell design offers significant flexibility. For example, it is possible to measure field components in two orientations (perpendicular to the laser beam) at the same time, and the full 3D magnetic field vector can be measured by splitting the laser beam and sending two beams through the same vapour cell. Even if single axis OPMs 12a-12c are used in the MEG system 100, their lightweight and flexible nature enables easy placement, meaning that they can be readily placed/mounted to measure field at different orientations.

Conventional SQUID and OPM-MEG systems are configured to measure only the radial components of magnetic field, as this is typically the component with the largest signal. However, as explained in more detail below, the measurement of magnetic field components in different directions/orientations across the sensor array 12 reduces the correlation between the contributions to the sensor data from the source of interest S1 within the subject's head H and a non-neuromagnetic field from an external source S2, which enables the contribution from the external source S2 to be better suppressed by the source reconstruction process (see below).

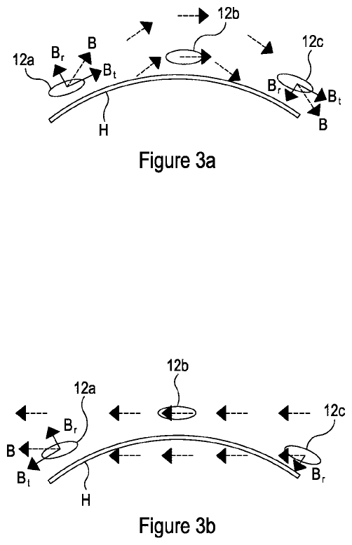

FIGS. 3a and 3b illustrate the general principle of the method 200. FIG. 3a shows a schematic magnetic field pattern (field vector B indicted by the dashed arrows) representative of that generated by a source of interest S1 in the head H. Assuming radially oriented sensors 12a-12c, sensor 12a would measure a radial field component B r directed out of the head (a positive field), sensor 12c would measure a radial field component B r directed into the head (a negative field), and sensor 12b would not detect any field. FIG. 3b shows a schematic of a very different magnetic field pattern which is substantially uniform field, representative of that generated by an external source S2. Because of the orientation of the radial sensors 12a-12c, again sensor 12a measures a positive field, sensor 12c a negative field, and sensor 12b nothing. This means, despite very different field patterns, the measured topography would be highly correlated. By contrast, if one of the sensors 12a-12c. e.g. sensor 12b, is rotated/arranged or configured to measure (instead or in addition) a tangential field component B t, it can readily be seen that the measurements made would indicate that the two different fields are in opposite directions. This would cause a reduction in their correlation, reducing the source reconstruction error. This is the basic premise of the effects described in sections 1 to 4 below.

In the following, the method 200 is validated by demonstrating theoretically and experimentally how a sensor array 12 according to the invention behaves when source localisation/reconstruction, using a beamformer spatial filter, is applied. Specifically, it is demonstrated that a MEG system 100 comprising single axis sensors 12a-12c and especially tri-axial sensors 12a-12c provides more accurate source reconstruction in the presence of interference from non-neuromagnetic fields.

### 1) Analytical Insights

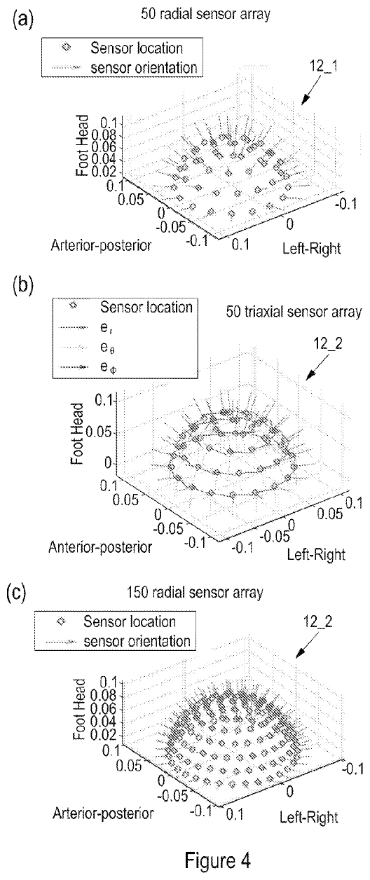

FIGS. 4a-4c show three hypothetical MEG sensor array configurations 12_1-12_3 considered. Array 12_1 comprises 50 radially oriented sensors (see FIG. 4a). Array 12_2 comprises 50 triaxial sensors in which each sensor provides three orthogonal measurements of magnetic field (giving 150 measurement channels in total) (see FIG. 4b). Array 12_3 comprises 150 radially oriented sensors (see FIG. 4c). In all three cases the sensors are assumed to be placed, equally spaced, on the surface of a sphere (of radius 8.6 cm). For the triaxial array 12_2, the sensors are oriented to measure magnetic field in the radial (r), polar (θ) and azimuthal (ϕ) orientations.

### 1.1) The Beamformer Spatial Filter

Source reconstruction is the process of deriving 3D images of electrical activity in the brain from measured magnetic field data. To understand how source reconstruction (and consequently MEG results) might differ across different designs of sensor array 12, a beamformer approach is used. Using a beamformer, the electrical activity, [symbol] (t) (units of Am), at some location and orientation, θ, in the brain is reconstructed based on a weighted sum of sensor measurements such that

[symbol]

(t)=w_{θ}^{T}b(t) [1]

where b(t) is a vector of the sensor data acquired across N measurement channels at time t, and the ‘hat’ notation denotes a beamformer estimate of the true activity, q_{θ}(t) (each sensor output for a given field orientation contributes a measurement channel to b(t)), w_{θ}^{T} is the transpose of a vector of weighting coefficients which would ideally be derived to ensure that any electrical activity originating at θ is maintained in the estimate, and all other activity suppressed (see Van Veen et al. “Beamforming: A versatile approach to spatial filtering”, IEEE ASSP Mag. 1988). To do this, the variance of the estimate (i.e. E ( [symbol] (t))²), where E(x) denotes the expectation value) is minimised subject to the linear constraint that source power originating at θ must remain. Mathematically, this is expressed as

min_{wθ}E(( [symbol] (t))²)subject to w_{θ}^{T}l_{θ}=1, [2]

where l_{θ} is a model of the magnetic fields that would be recorded in each measurement channel if there were a current dipole at θ with unit amplitude (i.e. l_{θ} is the forward model). The forward model contains the location and orientation of each sensor and channel. The solution to this is

$${w}_{θ}^{T} = \frac{{l}_{θ}^{T} {C}^{- 1}}{{l}_{θ}^{T} {C}^{- 1} {l}_{θ}} \qquad [ 3 ]$$

where C is the data covariance matrix.
1.2) A Single Source with Uncorrelated Gaussian Sensor Noise

We want to determine the error between the beamformer estimate [symbol] (t) and the true source timecourse q_{θ}(t) and how this is impacted by sensor array design. Initially, we start with the simplest possible case, where sensor data contain electrical activity from a single source (a SOI) S1 in the brain, with timecourse q(t), with the addition of random noise at each sensor, e(t). Here, the sensor data can be expressed as

b(t)=lq(t)+e(t), [4]

where l is the forward field for the source. We next assume that we use the beamformer to focus on the true location of the source, and that the source model is accurate (i.e. l_{θ}→l). In this case, a simple substitution of equations 3 and 4 into equation 1, and using an analytical form for the data covariance matrix (see M. J. Brookes el al. “Optimising experimental design for MEG beamformer imaging” Neuroimage 39, 1788-1802, 2008)) (see appendix A) allows us to write that

$$\hat{q} ( t ) = q ( t ) + \frac{1}{{‖ l ‖}^{2}} {l}^{T} e ( t ) . \qquad [ 5 ]$$

Where ∥l∥ is the Frobenious norm of the forward field vector from the source S1. Equation 5 shows that the source estimate [symbol] (t) contains a true representation of the source timecourse q(t) plus an error, which is the projection of the sensor noise through the forward field. Equation 5 only represents a single point in time and a more useful metric involves the summed square of the error in the reconstructed timecourse, across all time, which can be written as

$${E}_{t o t} = \frac{1}{\sqrt{M}} \sqrt{{\sum }_{i = 1}^{M} {( {\hat{q}}_{l} - {q}_{i} )}^{2}} , \qquad [ 6 ]$$

where M is the total number of time points in the sensor data recording. Mathematically, it can be shown (see Appendix A) that this total error in the beamformer reconstruction collapses to the convenient expression

$${E}_{t o t} = \frac{v}{‖ l ‖} . \qquad [ 7 ]$$

where υ is the standard deviation of the noise at each sensor 12a-12c, which we assume is equal across all sensors and is an inherent property, i.e. we shall assume to be fixed (at around 10 fT/√Hz for OPMs). ∥l∥ is a measure of how the sensor array is affected by the source S1, and it follows that, to minimise the overall error in the beamformer projected timecourse, the sensor array should be designed to maximise ∥l∥.

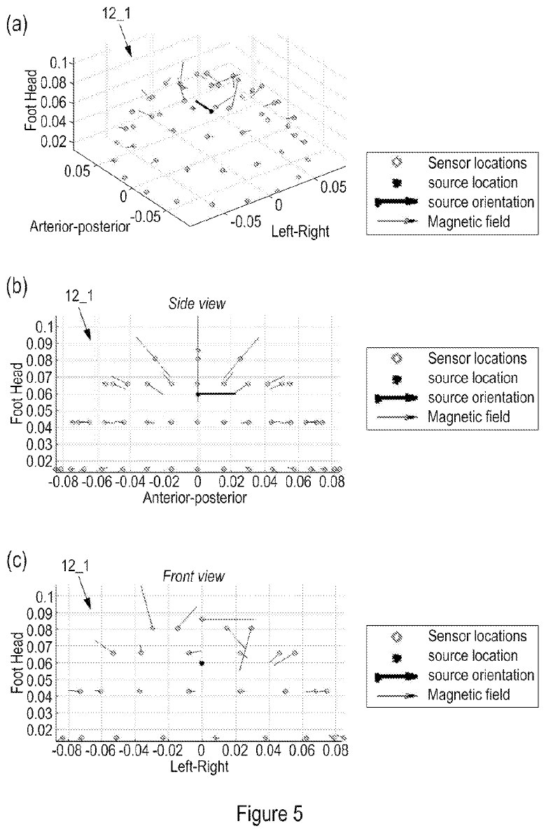

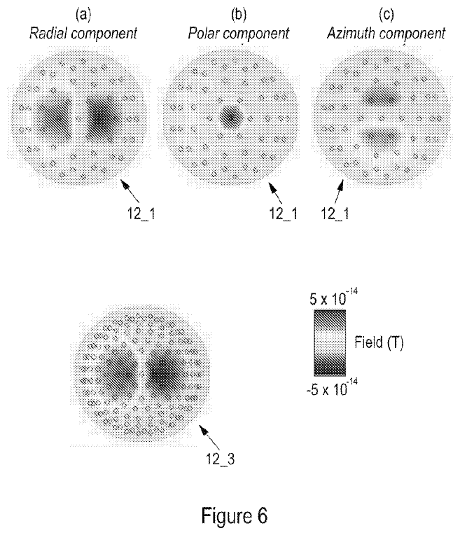

FIGS. 5-6 show how ∥l∥ behaves for each of the three sensor array configurations 12_1-12_3 in FIGS. 4a-4c. The magnetic field generated by a single source S1 in the brain was calculated at each sensor location within each of the sensor array configurations 12_1-12_3. The field was calculated based on the derivation by Sarvas (J. Sarvas “Basic mathematical and electromagnetic concepts of the biomagnetic inversion problem”, Physics in Medicine and Biology 32, 11-22, 1987) assuming the head H to be approximated by a spherically symmetric homogeneous conductor and that the source S1 of brain electrical activity can be approximated as a current dipole. The forward field/was calculated at the sensor locations as the dot product of the field vector B with the sensor detection axes. Note that for the triaxial array 12_2, l is composed of the fields for the three orientations (i.e. I=[l_{radial}, lₚₒₗₐᵣ, l_{azimuth}]). This calculation was run 25000 times, with the source S1 in a different location in the brain each time.

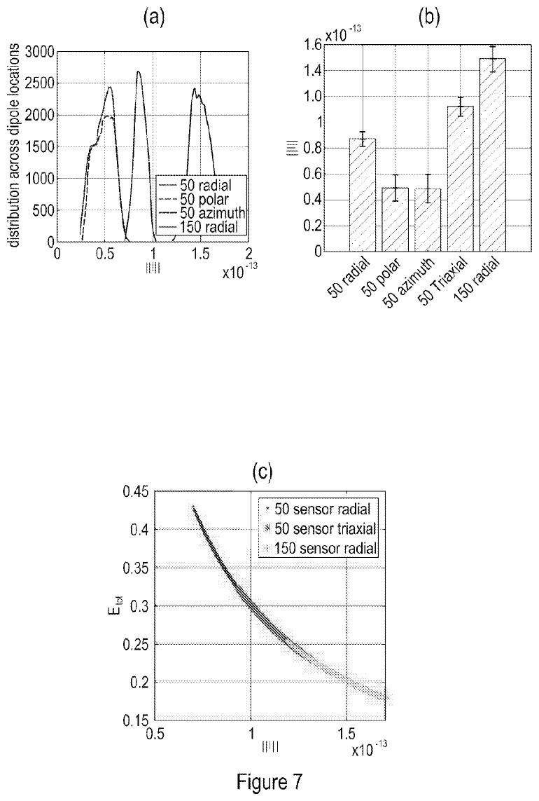

FIGS. 5a-c show an example magnetic field vector B computed at each sensor location in the 50-sensor radial array 12_1 generated by a single source S1, viewed from three different orientations. FIGS. 6a-c show maps of the spatial distribution of the radial B_{rad}, polar Bₚₒₗ and azimuthal B_{azi} field components of the vector field B shown in FIGS. 5a-c on a flattened representation of the head H (the open circles indicate sensor locations). For comparison, the distribution of radial fields B_{rad} for the 150-sensor radial array 12_3 is also shown in FIG. 6d. FIGS. 7a and 7b show the histograms of the ∥l∥ values, and the mean ∥l∥ values across all realisations of source S1 location, respectively. As seen, ∥l∥ is higher in the radial orientation than in the tangential orientations (polar and azimuthal) due to the generally higher signal in the radial direction. FIG. 7c shows the dependence of the total error against ∥l∥ values across all realisations of source S1 locations for each array 12_1, 12_2, 12_3 following the trend of equation 7. Consequently, ∥l∥ for a 50-sensor triaxial array 12_2 (with 150 measurement channels) is higher than for a 50-sensor radial array 12_1 (as one would expect given the increased channel count), but not as high as for a 150-sensor radial array 12_3. Equation 7 therefore shows that the total source reconstruction error can be reduced by adding triaxial sensors, but not by the same degree that it would be if we used 150 radial sensors.

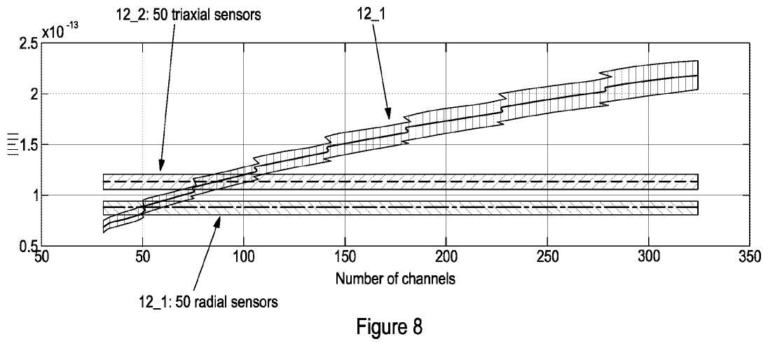

FIG. 8 shows (red line) how ∥l∥ varies with sensor count for an array 12_1 of radially oriented sensors (the shaded area indicates standard deviation). An algorithm was used to place between 31 and 325 sensors equidistantly on a sphere surface. For each sensor count, we simulated 25 source locations and computed the average value of ∥l∥. As expected, ∥l∥ increases approximately monotonically with sensor count (the discontinuities are due to the way in which the algorithm placed the sensors on the sphere). The mean value of ∥l∥ for a 50-sensor radial array 12_1 (blue line), and a 50-sensor triaxial array 12_2 (black) is also shown for comparison, where it can be that ∥l∥ for the 50-sensor triaxial array 12_2 is approximately equal to that for an 80-sensor radial array.

1.3) Two Sources with Uncorrelated Gaussian Sensor Noise

The analysis in section 1.2 is oversimplified because, generally, one has more than one “active” source contributing to the measured magnetic field at each sensor location. In the following we examine a mathematical model with two active sources: a first source S1 (the SOI) in the brain with timecourse q₁(t) and forward field l₁; and a second source S2 with timecourse q₂(t) and forward field l₂ representing interference e.g. a source outside the brain. In this scenario, the sensor data, b(t), are given by

b(t)=l₁q₁(t)+l₂q₂(t)+e(t) [9]

where again e(t) contains the sensor errors/noise. As before we assume that we reconstruct a source at the true location and orientation of source 1 so that,

[symbol]

(t)=w₁^{T}(l₁q₁(t)+l₂q₂(t)++e(t))=q₁(t)+w₁^{T}l₂q₂(t)+w₁^{T}e(t). [9]

Note that, as with equation 5, the estimate of the timecourse of source 1 ( [symbol] (t)) again contains the true source timecourse (q₁(t)) but now with two sources of error. For convenience we rewrite equation 9 as

[symbol]

(t)=q₁(t)+δq₂(t)+ϵ(t), [10]

and it is easy to see that the term δq₂(t) represents interference from source S2 whilst ϵ is the error introduced by sensor noise. As such, in designing a MEG sensor array 12 both terms should be minimised.

Again by exploiting the analytical formulation of the data covariance matrix it can be shown (see Appendix B) that

$$δ = \frac{‖ {l}_{2} ‖}{‖ {l}_{1} ‖} {r}_{12} [ \frac{1 - {f}_{2}}{1 - {f}_{2} {r}_{12}^{2}} ] \qquad [ 11 ]$$

where

$${r}_{2} = \frac{{l}_{1}^{T} {l}_{2}}{‖ {l}_{1} ‖ ‖ {l}_{2} ‖} . \qquad [ 12 ]$$

The quantity f₂ represents a scaled signal to sensor noise ratio for field from source S2 and is given by

$${f}_{2} = \frac{{Q}_{2}^{2} {‖ {l}_{2} ‖}^{2}}{{v}^{2} + {Q}_{2}^{2} {‖ {l}_{2} ‖}^{2}} \qquad [ 13 ]$$

where Q₂ is the standard deviation of q₂(t) across the duration of the MEG sensor data recording. Note that for very high signal to noise ratio, f₂→1 and for very low signal to noise ratio f₂→0. Here r₁₂ is a measure of the similarity of the respective lead field patterns l₂ and l₂ for sources S1 and S2. Geometrically this quantity represents the cosine of the angle between the vectors l₁ and l₂. Statistically it is directly related to the Pearson correlation coefficient between the two forward fields l₂ and l₂. For example, on the one hand, if sources S1 and S2 were completely inseparable (l₁=l₂) then r₁₂=1. On the other hand, if l₁ and l₂ look completely different (e.g. as might be the case if sources S1 and S2 were both brain sources on opposite sides of the head) then r₁₂=0. Note in this latter case, the interference from source S2 falls to zero.

Equations 10 and 11 are important since it tells us how a beamformer separates two sources S1, S2. It governs spatial resolution (i.e. our ability to separate multiple sources in the brain, and it also highlights the advantages of beamforming over, e.g. a dipole fit (see Appendix C).

Using a similar mathematical approach it is also possible to derive an expression for the error on the signal due to sensor noise. Specifically it can be shown (see appendix B) that

$$ϵ ( t ) = \frac{v}{‖ {l}_{1} ‖} [ {r}_{1 e} ( t ) ( \frac{1}{1 - {f}_{2} {r}_{12}^{2}} ) - {r}_{2 e} ( t ) ( \frac{{f}_{2} {r}_{12}}{1 - {f}_{2} {r}_{12}^{2}} ) ] \qquad [ 14 ]$$

where

$${r}_{1 e} ( t ) = \frac{{l}_{1}^{T} e ( t )}{‖ {l}_{1} ‖ v}$$

denotes spatial correlation between the forward field l₂ of source S1, and the sensor noise pattern e(t). Similarly

$${r}_{2 e} = \frac{{l}_{2}^{T} e ( t )}{‖ {l}_{2} ‖ v}$$

denotes spatial correlation between the forward field l₂ of source S2, and the sensor noise pattern e(t).

This analytical description of additive sensor noise on a beamformer source reconstruction is only valid for a single time point and so, as previously, it is useful to quantify the total error across an entire timecourse (measurement acquisition time). To do this we again use the sum of the squared difference between the reconstructed and original timecourse, thus

Eₜₒₜ²=Σₜ₌₁^{M}( [symbol] −q₁ᵢ)² [15]

Where the index i denotes the time point and M is the total number of time points in the sensor data recording. As shown in appendix B, assuming that sensor noise and both the timecourses of both sources S1, S2 are temporally independent, the total error on the timecourse (Eₜₒₜ²) is given by the sum of the error from source S2, and the error from sensor noise, according to

$${E}_{tot}^{2} = {E}_{source}^{2} + {E}_{n o i s e}^{2} \qquad [ 16 ]$$

$$where$$

$${E}_{source}^{2} = \frac{{Q}_{2}^{2} {‖ {l}_{2} ‖}^{2}}{{‖ {l}_{1} ‖}^{2}} {{r}_{12}^{2} [ \frac{1 - {f}_{2}}{1 - {f}_{2} {r}_{12}^{2}} ]}^{2} \qquad [ 17 ]$$

$$and$$

$${E}_{noise}^{2} = \frac{{v}^{2}}{{‖ {l}_{1} ‖}^{2}} ( \frac{1 + {f}_{2}^{2} {r}_{12}^{2} - 2 {f}_{2} {r}_{12}^{2}}{{( 1 - {f}_{2} {r}_{12}^{2} )}^{2}} ) \qquad [ 18 ]$$

Note that in the case where either source S2 does not exist (f₂=r₁₂=0) or where the two sources S1, S2 are separable/do not correlate (r₁₂=0) then the interference from the second source S2 collapses to zero, and

$${E}_{n o i s e}^{2} = \frac{{v}^{2}}{{‖ {l}_{1} ‖}^{2}}$$

as before for the single source case.

The above analysis shows that beamformer accuracy is governed by a relatively small number of parameters, some of those parameters are invariant to system design: e.g. Q₁ is set by the brain; Q₂ by the nature of the interference source S2; and υ is inherent to the sensor. However, other parameters can be altered by the way in which the sensor array 12 is configured. For example, as shown in FIGS. 8, ∥l₁∥ and ∥l₂∥ will both increase with channel count and ∥l₁∥ will typically be larger for radially oriented sensors. More importantly, the correlation of field topographies (r₁₂) from the different sources S1, S2 can be altered by judicious sensor array design. For this reason, an understanding of equations 17 and 18, to appreciate how these parameters relate to overall MEG source reconstruction accuracy, becomes important.

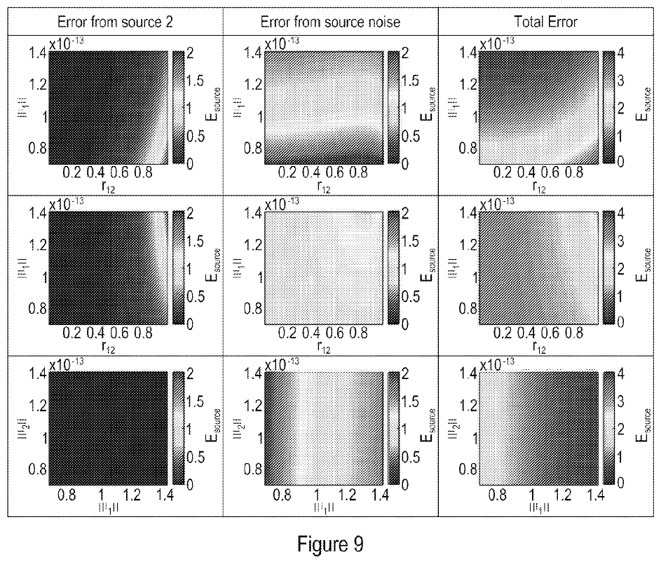

FIG. 9 shows a visualisation of equations 17 and 18 for a realistic range of values for ∥l₁∥ and ∥l₂∥ for the three array configurations 12_1-12_3 in FIG. 4. The sensor noise, υ, was set to 100 fT and both source amplitudes (Q₁ and Q₂) were set to 1 nAm. The left, centre and right columns show the errors from interference from source S2, sensor noise, and the total error respectively. The upper row shows how error behaves when varying ∥l∥ and r₁₂. The middle row shows error versus ∥l₂∥ and r₁₂. Finally the lower row shows error versus ∥l₁∥ and ∥l₂∥. Note that, for a fixed value of r₁₂, error decreases monotonically with increasing ∥l₁∥, while varying ∥l₂∥ has relatively little effect.

FIG. 9 shows that the two important parameters to minimise total beamformer error are ∥l₁∥ and r₁₂. If a sensor array 12 can be optimised such that r₁₂ is minimised, whilst ∥l₂∥ is maximised, this can result in a huge reduction in overall error.

To understand how r₁₂ relates to sensor array design r₁₂ was calculated for the three different sensor array configurations 12_1-12_3 shown in FIG. 4 using a model of a current dipole in a conducting sphere as before. One source S1 (the SOI) in the brain and one source of interference S2 was simulated. Source S1 was simulated at a depth of between 2 cm and 2.4 cm from the sphere surface and with (a randomised) tangential orientation. Two types of interference source S2 were considered, a source of interference internal and external to the brain. The internal source of interference comprised a current dipole within the conducting sphere (which would model a second source of no interest in the brain) and was also tangentially oriented (randomly). The distance between the source of interest S1 and internal interference source S2 was derived from a uniform distribution, and was between 2 and 6 cm. For convenience, the external source of interference S2 was also taken to be a current dipole and was located between 20 cm and 60 cm from the centre of the sphere. r₁₂ was calculated for both internal and external interference sources S2. 25,000 iterations of this calculation were run with the source locations S1, S2 changing on each iteration.

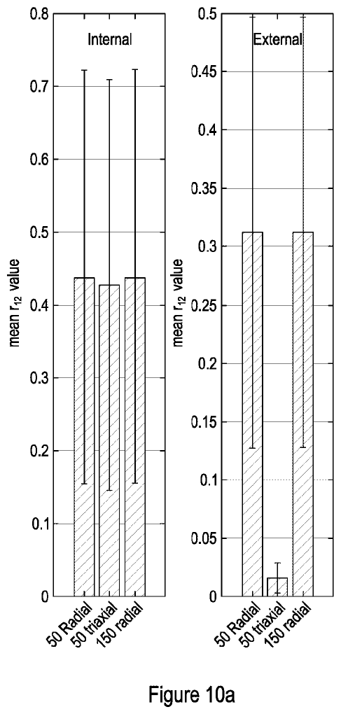

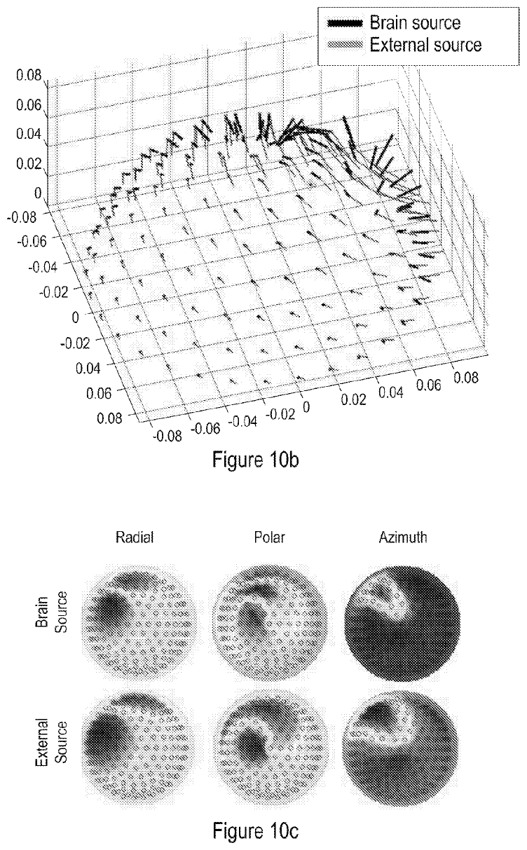

FIG. 10a shows calculated r₁₂ values averaged over iterations for internal (left) and external (right) interference sources for each of the three sensor array configurations 12_1-12_3. For internal sources of interference S2, the improvement offered by a triaxial sensor array 12_2 over the radial sensor arrays 12_1, 12_3 is modest. By contrast, for external sources of interference S2 the improvement is dramatic. The reason for this is summarised in FIGS. 10b and 10c. FIG. 10b shows a single example of the magnetic field vectors present at each sensor of the 150-sensor array 12_3 from the internal/brain source S1 (black) and an external source S2 (blue). As shown, the vector fields differ dramatically. However, when just taking the radial projection of these field vectors, which is shown in the left-hand radial field distribution maps of FIG. 10c, the two field patterns look similar. The field patterns for the two tangential components (polar and azimuthal field projections) look similar also, but whilst the radial components are positively correlated, both of the tangential components are negatively correlated. This means that when the radial, polar and azimuthal projections are concatenated (combined) correlation will be reduced compared to any one projection alone. Whilst this is only one example, it illustrates the reason why the value of r₁₂ is reduced in the triaxial sensor array 12_2 compared to the two simulated radial sensor arrays 12_1, 12_3. This concept is discussed further below.

As such, the addition of triaxial sensors has a dramatic effect on r₁₂ for sources of interference S2 outside the brain. Consequently, particularly in cases where large external interference is expected, a triaxial sensor array 12_2 will offer a marked advantage over a radial sensor array 12_1, 12_3, even if the latter has a very high channel count. This theory provides the basis for the simulations presented in the next section.

### 2) Simulations

### 2.1) Effect of Interference on Beamformer Reconstruction

Based on our analytical insights derived in the preceding sections, with no interference, a 150-sensor radial array 12_3 should outperform a 50-sensor triaxial array 12_2 (a consequence of the higher forward field norm). However, as soon as interference is introduced external to the brain the triaxial system offers improved source reconstruction performance due to its ability to better separate source topographies. In the following, the effect of the three sensor array configurations 12_1-12_3 shown in FIG. 4 on beamformer source reconstruction is simulated.

For all simulations a spherical volume conductor head model was used. The source, interference and sensor noise were simulated as follows:
Source simulation: A single source S1 (the SOI) in the brain was simulated. The internal source S1 was located between 2 cm and 2.4 cm from the head (sphere) surface to mimic activity in the cortex. Apart from depth, the source location within the head H was random. Source orientation was tangential to the radial direction (as is commonly found in the brain), but otherwise random. The internal source timecourse q₁(t) comprised Gaussian distributed data sampled at 600 Hz, and the root-mean-square amplitude was set to 1 nAm. The forward field l₁ was based on a current dipole model as is common and well known in the art.
Interference simulation: As before, sources of interference external and internal to the brain were simulated (the former representing e.g. laboratory equipment and the latter representing ‘brain noise’).
   For external interference, 80 sources of magnetic field were generated at distances ranging from 20 cm to 60 cm from the centre of the sphere/head H. Interference source timecourses q₂(t) comprised Gaussian distributed random data and their locations were randomised. The interference sources S2 were assumed to be current dipoles (each embedded within its own conducting sphere) oriented perpendicular (tangential) to the vector/line joining the centre of the head to the dipole location. The interference source strength/amplitude, Q₂, was calculated in proportion to the strength/amplitude of the internal source of interest S1, Q₁. Specifically, the interference amplitude was set as

$${Q}_{2} = α {Q}_{1} \frac{‖ {l}_{1} ‖}{‖ {l}_{2} ‖}$$

where α controls the relative strength of interference source S2.
For internal interference, 15 current dipoles were simulated in the head H. Interference source timecourses q₂(t) were Gaussian distributed random data, and the source amplitudes were set in proportion to the source of interest S1 as with the external interference sources. Unlike the source of interest S1 which was constrained to the cortex, internal interference sources S2 could take any location in the head H but were a between 2 cm and 6 cm from the source of interest S1 (Euclidean distance) and orientated tangentially.
Additive noise: Sensor noise was assumed to be Gaussian distributed random noise, independent, but with equal amplitude, across sensors. This was added with an amplitude of 30 fT.

A total of 300 seconds of sensor data were simulated in this way. For each iteration of the simulation different source and interference locations were used and a took values ranging from 0 to 1.4 in steps of 0.1 to increase the impact of interference on the sensor data (different source/interference timecourses, and a different realisation of noise was used for each a). 25 iterations of the simulation were run. Source and interference timecourses q₁(t), q₂(t) were the same for each sensor array configuration 12_1-12_3 although different (random) sensor noise was used for the three configurations. Each dataset, for each array configuration 12_1-12_3, was processed using a beamformer, as described above. Prior to beamforming, we simulated a co-registration error on the sensor locations such that the location and orientation of the sensors used for beamforming were not the same as those used to simulate the data. Specifically, sensor locations and orientations underwent a 2 mm translational, and 2 mm rotational affine transformation whose direction was randomised. This type of co-registration error is similar to what would be observed experimentally. Data covariance was calculated in the 0-300 Hz frequency window, and a time window encompassing the full 300 second simulation. No regularisation was used.

To image the source, a pseudo-Z-statistic approach was used, which contrasts beamformer projected power to noise. The pseudo-z-statistic represents a “signal power to noise” measurement. Mathematically, the signal power is given by W^{T}*C*W where W are the weights and C the data covariance matrix. The noise power is given by W^{T}*S*W where S is the noise covariance matrix (which is usually taken to be the identity matrix multiplied by a scalar representing the noise variance of a sensor). Z is one divided by the other. Images were generated within a cube with 12 mm side length, centred on the true location of source S1. The cube was divided into voxels (of isotropic dimension 1 mm) and for each voxel the source orientation was estimated using the direction of maximum signal to noise ratio. A single image was generated per simulation. In each case, the peak pseudo-Z statistic was found and its location used to reconstruct the timecourse of peak electrical activity in the cube. Three measures of beamformer accuracy/performance are derived.
1. Localisation error: The location of the peak electrical activity in the beamformer image is found and its displacement from the true source location computed. This provided a measure of localisation error.
2. Timecourse error: The sum of squared differences between the reconstructed timecourse (at the location of the peak in the beamformer image) and the true timecourse is computed.
3. Timecourse correlation: The temporal Pearson correlation between beamformer reconstructed source timecourse and the true timecourse is computed (at the location of the peak in the beamformer image).

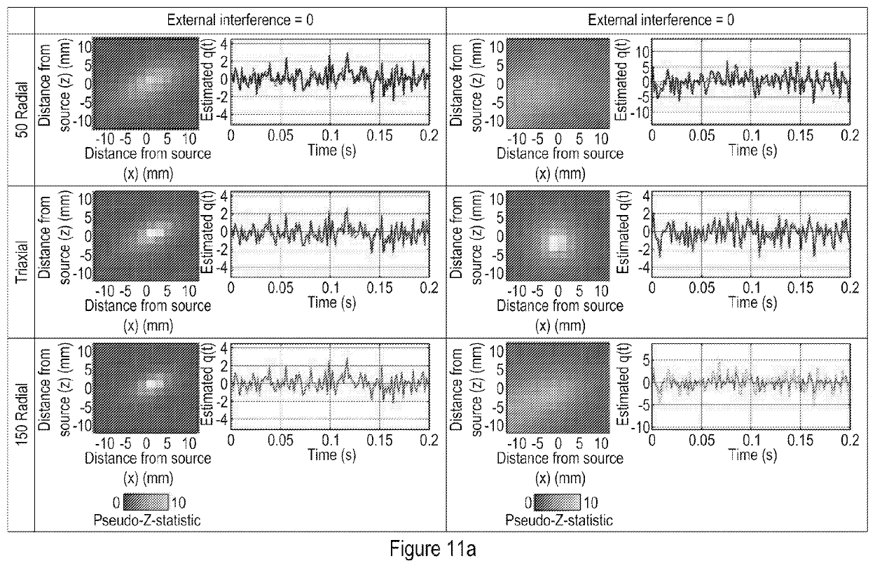

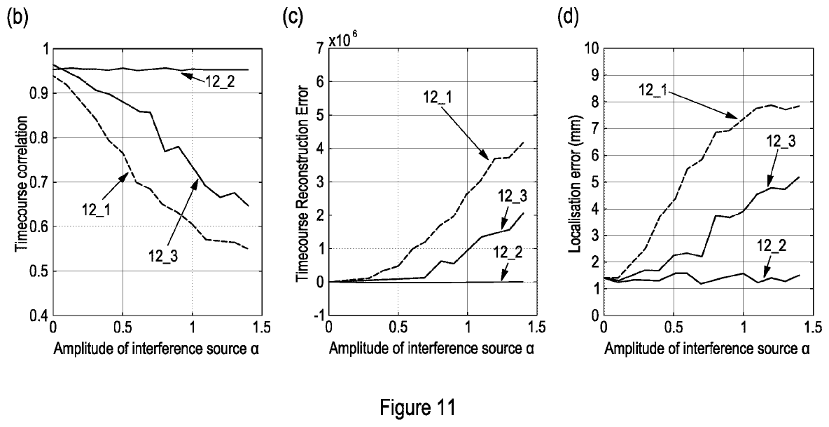

FIG. 11a shows example beamformer images and reconstructed timecourses for the three sensor array configurations 12_1-12_3 for external source interference. The left-hand panel shows the results for no external source interference (α=0) and the right-hand panel shows the results including external interference sources S2, each with amplitude equivalent to that of the source of interest S1 (α=1). In both cases the top centre and bottom panels show results for the 50-sensor radial array 12_1, the 50-sensor triaxial array 12_2 and the 150-sensor radial array 12_3 respectively. As expected with no external interference all three sensor arrays 12_1-12_3 faithfully reconstruct the source of interest S1 (the small localisation error likely results from the simulated co-registration error). However, when external interference is added, for both radial sensor arrays 12_1 and 12_3 the beamformer image and the source timecourse reconstruction are degraded. By contrast, the triaxial array 12_2 maintains a faithful reconstruction of the source S1.

FIGS. 11b, 11c and 11d show the corresponding timecourse correlation, timecourse error and localisation accuracy for each sensor array 12_1-12_3 with external source interference, as a function of the interference amplitude in terms of cc. In both of the radial sensor arrays 12_1, 12_3 reconstruction accuracy/performance degrades as interference is added. By contrast, the triaxial sensor array 12_2 remains unaffected by the external interference. Note that, with no interference, the 150-sensor radial array 12_3 outperforms the triaxial array 12_2 as expected due to the increased channel count. However, as soon as external interference is introduced, the triaxial array 12_2 gains a significant advantage.

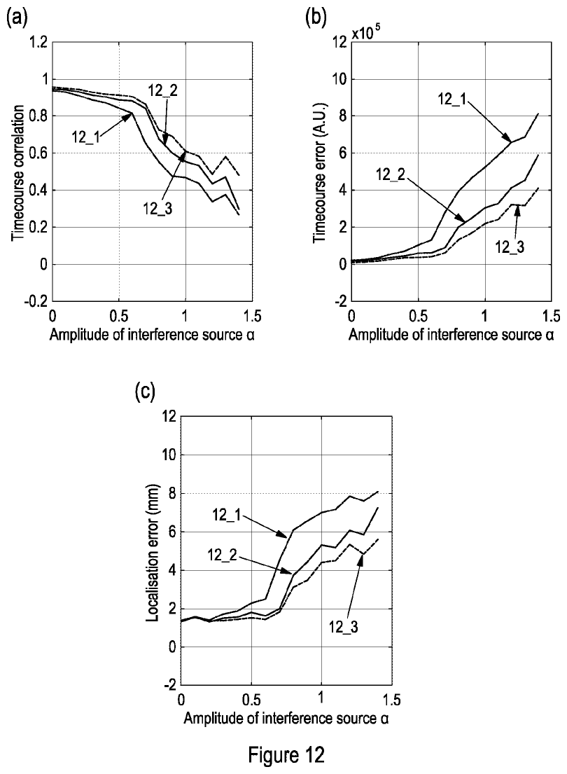

FIGS. 12a-12c show the corresponding timecourse correlation, timecourse error and localisation accuracy for each sensor array 12_1-12_3 with internal source interference, as a function of the interference amplitude in terms of α. Here, the measurement of vector fields with a triaxial sensor array does not significantly help to distinguish between sources and consequently, a triaxial sensor array offers less improvement.

### 2.2) Effect of Head Movement on Beamformer Reconstruction

In principle, a motion artifact behaves somewhat like external interference. However, unlike external sources S2 of interference, which typically results in a spatially static field, movement artifacts manifest as an apparently moving field.

To simulate motion artifacts, we first generated a set of movement parameters. As with any rigid body, we assumed six degrees of freedom for the simulated helmet/head: translation in x, y and z, and rotation about x, y and z. For each degree of freedom, we simulated a ‘motion time series’ which collectively would define how the helmet moved relative to a static background field. The motion time series comprised Gaussian distributed random data which were frequency filtered to the 4 to 8 Hz frequency band (movement is assumed to be mostly low frequency). Each of the six motion time-series comprised a single common signal (i.e. modelling movement about multiple axes at the same time) and a separate independent signal (i.e. modelling temporally independent movements on each axis). The amplitude of the common signal was 5 mm translation and 3° rotation, and the amplitude of the independent signal was 2 mm translation and 2° rotation. Following construction of the motion time series, the motion was applied to the helmet via affine transformation.

We assumed three different conditions for the background field. 1) No field (i.e. so movement will have no effect). 2) A static and uniform background field of B(r)=[5 5 5] nT where r represents position (i.e. rotations will cause artifacts, but translations will have no effect). 3) A static but non uniform background field. Here B(r)=Bₒ+G. r where Bₒ (=[5 5 5] nT) is a spatially uniform background field and G is a 3×3 matrix which describes the linear magnetic field gradients. For the simulation we assumed

$$G = [ \begin{matrix}10 & 5 & 8 \\ 5 & 10 & 5 \\ 8 & 5 & - 10\end{matrix} ] {nTm}^{- 1} \qquad [ 19 ]$$

The reflectional symmetry in the matrix is imposed by the Maxwell equations. For each time point, the location and orientation of every sensor in the helmet was calculated according to the motion time series, and the local field vector calculated. The field ‘seen’ by each sensor was estimated as the dot product of the sensor detection axis(es) with the field vector, B(r).

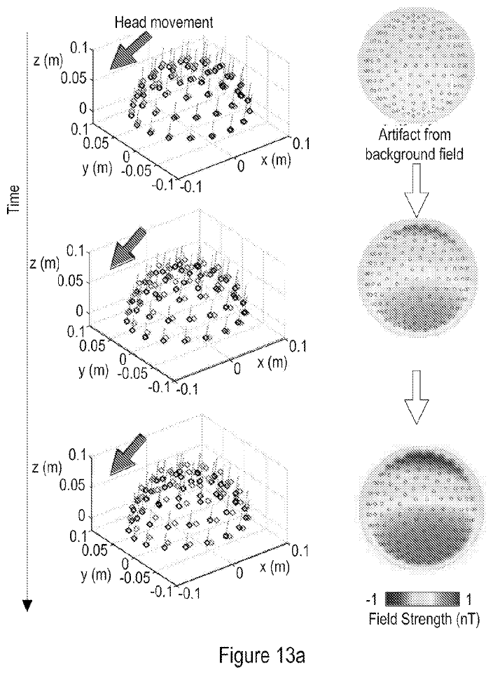

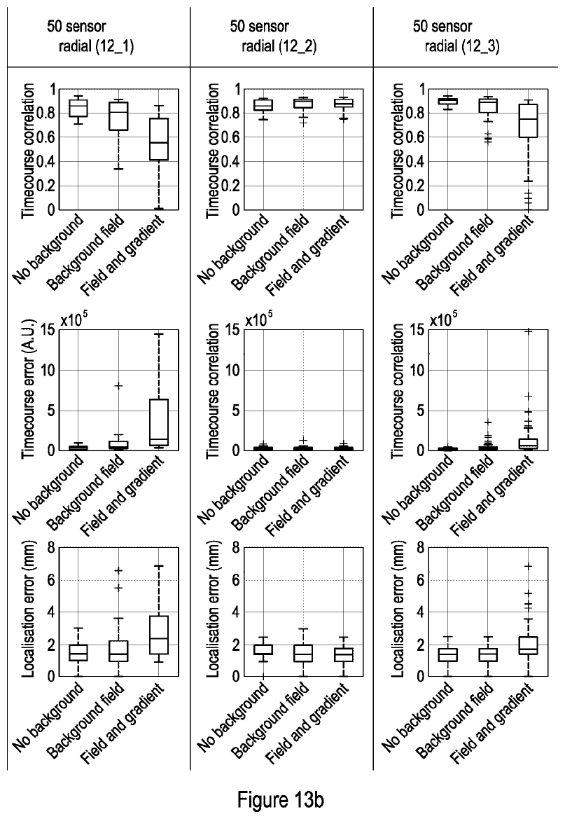

OPM sensors come equipped with on-board electromagnetic coils configured to zero the field at the measurement location (this is a requirement since OPMs are designed to operate close to zero field). This means that, at the start of a MEG experiment (i.e. with the head/helmet in its starting position) the fields measured by an OPM sensor array 12 will be zero. At this point, the current in the on-board coils is locked. To simulate this, the artifact was assumed to be the measured field shift between the first timepoint, and all other timepoints. An example of this process is shown in FIG. 13a, for a 50-sensor radial array 12_1.

A single dipolar source of interest S1 was simulated at a depth of between 2 cm and 4.8 cm from the surface of the spherical conductor, with 1 nAm amplitude as before. The source S1 was tangentially oriented and its location randomised. The source timecourse comprised Gaussian distributed random noise, which was frequency filtered to the 4-8 Hz band to mimic a situation where the source of interest S1 is obfuscated (in terms of frequency) by the movement artifact. Gaussian distributed random sensor noise was added with an amplitude of 30 ff, which was also frequency filtered to the 4-8 Hz band. For each of the three separate background field conditions, the simulation was run 50 times with a different location of the source of interest S1 on each iteration. To assess the extent to which the beamformer can reconstruct the source of interest S1 we again measured timecourse correlation, timecourse reconstruction error, and localisation error. The results are shown in FIG. 13b.

In FIG. 13b, the measured timecourse correlation, timecourse reconstruction error, and localisation error are shown in the three rows. The left, centre and right columns show results for the 50-sensor radial array 12_1, the 50-sensor triaxial array 12_2, and the 150-sensor radial array 12_3 respectively. Consistent with the results in FIG. 11 for external source interference, the reconstruction performance of the two radial arrays 12_1, 12_3 degrades as the motion artifact is added, and made more complex. As would be expected from the greater channel count, the 150-sensor radial array 12_3 performs better than the 50-sensor radial array 12_1. However, the triaxial array 12_2 outperforms both radial arrays 12_1, 12_3, with little or no loss in reconstruction performance as the motion artifact is added.

2.3) A MEG System with Mixed Sensor Orientation

The above simulations results demonstrate the theoretical advantages of using a triaxial sensor array 12_2 in a MEG system 100 over a traditional radial sensor array 12_1, 12_3. In particular, the ability to better distinguish sources of interference external to the brain from the neuromagnetic field of interest means that the triaxial array 12_2 is much less affected once source reconstruction has been applied. In a similar way, if a wearable OPM triaxial sensor array is used in which a subject's head H moves during a MEG measurement, by rotating and/or translating their head H in a background field, the resulting motion artifact can be better suppressed by a triaxial sensor array compared to a radial only array.

It will be appreciated that the same principle applies to a dual-axial sensor array where each sensor measures field along a radial axis r and one tangential axis t (polar or azimuth), and also to a single axis sensor array where just a small number of single axis sensors are arranged to measure field along a tangential axis t (polar or azimuth), as shown below. The fundamental principle is that measuring field in different orientations helps to differentiate sources of magnetic field outside the brain by reducing r₁₂.

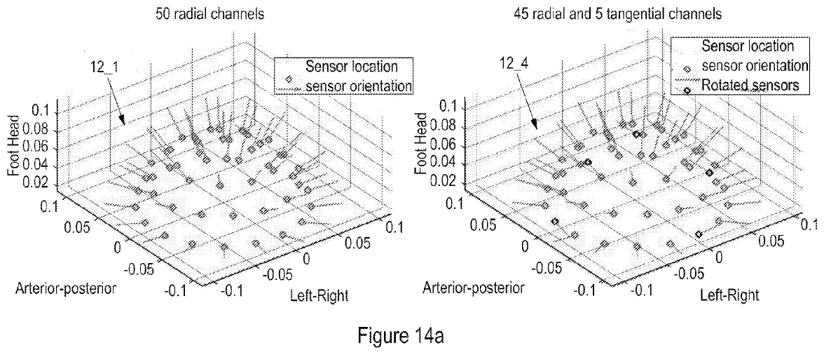

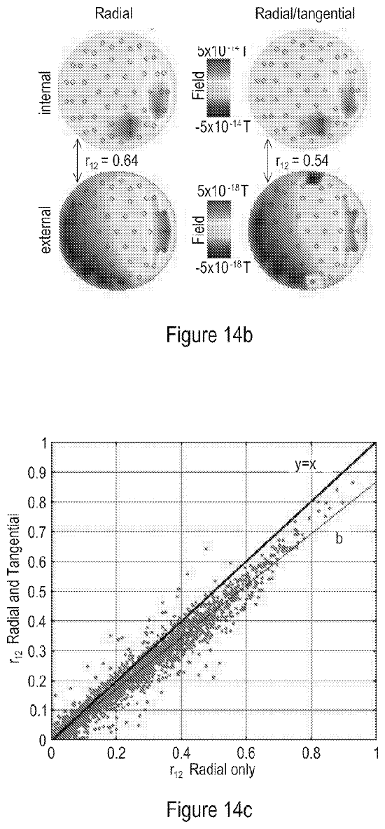

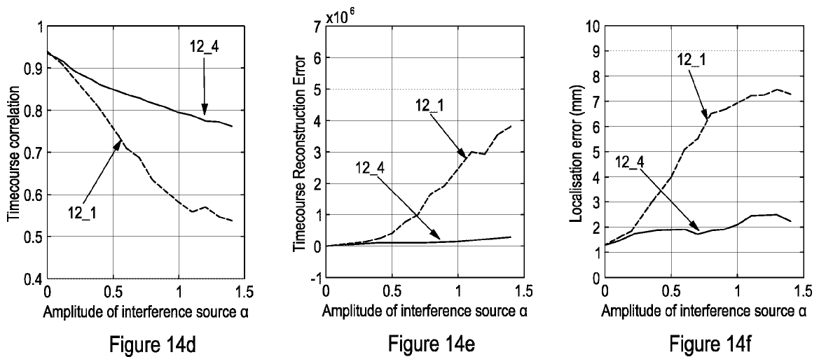

FIG. 14a shows a simulated 50-sensor radial array 12_1 and a 50-sensor “mixed” array 12_4 where a small number (five) of sensors (indicated by the black open circles) have been rotated/arranged to measure tangential field. The sensor locations are identical in both sensor arrays 12_1 and 12_4. For both sensor arrays 12_1 and 12_4, a source of interest S1 in the brain is simulated in 25 different locations (dipolar, oriented tangentially and location randomised, as before). For each internal source S1 we simulated 80 sources S2 of external interference (also current dipoles, at a distance between 20 cm and 60 cm from the centre of the head).

FIG. 14b shows an example of the field distribution at the sensor locations for one source pair (internal source S1 and external interference source S2) for the 50-sensor radial array 12_1 and the 50-sensor mixed array 12_4. Notably, the external interference source S2 topography is altered by the sensor rotation and this leads to a drop in the r₁₂ value from 0.64 to 0.54, as shown. For each source pair realisation, the correlation between their spatial topographies (i.e. r₁₂) was calculated. FIG. 14c shows all r₁₂ values for the 50-sensor radial array 12_1 plotted against the equivalent r₁₂ values for the 50-sensor mixed array 12_4. If the rotation of sensors in array 12_4 had no effect, then these values would fall along the y=x line (shown in black). However, they consistently fall beneath it (line of best fit is shown in blue by line b), implying that r₁₂ is, on average, lowered by the rotation of sensors. Whilst this effect is marginal in this example, because beamformer estimated error is a non-linear function of r₁₂ (see equation 17) even a marginal reduction in r₁₂ will yield a relatively large improvement in beamformer reconstruction performance.

FIGS. 14d-14f show the corresponding source reconstruction performance metrics: timecourse correlation, timecourse error and localisation accuracy for two sensor arrays 12_1 and 12_4 with external source interference, as a function of the interference amplitude a. It can be seen that even rotating five sensors in a 50-sensor array to measure tangential field has a relatively large effect, with a significant improvement in source reconstruction performance; although not as dramatic as seen for the full triaxial sensor array 12_2 (compare FIGS. 14d-f with FIGS. 11b-11d).

### 3) Experimental Verification

In the following the triaxial sensor “principle” is experimentally validated using an single axis OPM sensor array 12 comprising a first plurality of OPM sensors arranged to measure field along a radial axis r and a second plurality of OPM sensors arranged to measure field along a tangential axis t. This was achieved essentially by taking a radial only sensor array and rotating the second plurality of OPM sensors by 90°. A single subject (male, aged 25 years, right handed) took part in the experiment, which was approved by the University of Nottingham Medical School Research Ethics Committee. On each trial of the experiment (performed in an MSR 40), the participant was shown a visual stimulus (a black and white horizontal grating projected onto a screen inside the MSR 40) for 2 seconds, followed by 3 second rest period with no stimulus. Whilst the stimulus was on the screen, the participant was asked to make continuous abductions of their left index finger. The experiment comprised 50 trials and was repeated 4 times. This paradigm gives a robust response in the beta (13-30 Hz) frequency band.

Sensor data were recorded using a 45-sensor array of single axis OPMs (i.e. 45 measurement channels). This sensor array has been described in R. M. Hill et al. “Multi-channel whole-head OPM-MEG: Helmet design and a comparison with a conventional system” Neuroimage 219, 116995, 2020). Figure shows the two experimental sensor arrays 12_5r, 12_5m. In the left hand sensor array 12_5r all 45 sensors are oriented to detect radial field, and the in the right hand sensor array 12_5m 40 sensors are oriented to detect radial field and 5 sensors are oriented to detect a tangential field (i.e. rotated through compared to sensor array 12_5r). The OPM sensors 12a-12c were manufactured by QuSpin Inc. formulated as magnetometers, and mounted on a 3D printed rigid helmet (not shown). Their location and orientation with respect to brain anatomy was found using a combination of the known geometry of the 3D printed helmet (which gives sensor locations and orientations relative to the helmet, and each other) and a head digitisation procedure, based upon optical scanning (see same R. M. Hill et al. 2020 reference) which provides a mapping of the helmet location to the head. In the first and third run of the experiment, the radial only sensor array 12_5r was used, and in the second and fourth runs of the experiment, the mixed sensor array 12_5m was used. This gave a similar experimental setup to that simulated in FIG. 14.

Sensor space analysis: Sensor space refers to the actual measured sensor data. Sensor data were frequency filtered into the beta band (12-30 Hz) and segmented into the separate trials. For each sensor, and each trial, data were Fourier transformed to provide an amplitude spectrum in order to visualise how the beta-band data were contaminated by artifacts (discussed further below).

Source space analysis: Source space refers to the reconstructed timecourse and location of the source of interest. Sensor data were projected into source space using a beamformer according to equations 1 and 2. The sensor data covariance was computed in the beta band. Sensor data were segmented into trials and, in order to avoid discontinuities between trials affecting the covariance estimate, a separate covariance matrix C was calculated for each trial, and the average over trials was used. No regularisation was applied. The forward field l₁ was based on a spherical volume conductor model, using the best fitting sphere to the subject's head shape, and the dipole approximation for the source of interest. Data were reconstructed to 78 locations in the cortex which each corresponded to the centroid of a cortical region, defined based on the Automated Anatomical Labelling (AAL) brain atlas. For each region, the Fourier transform of reconstructed timecourse data in each AAL region was computed, for each trial. Associated amplitude spectra were derived and averaged across trials and regions. This analysis was applied to each of the four experimental runs, independently.

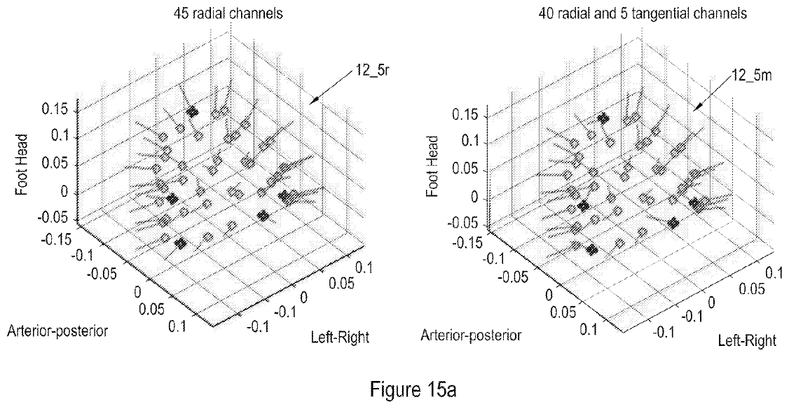

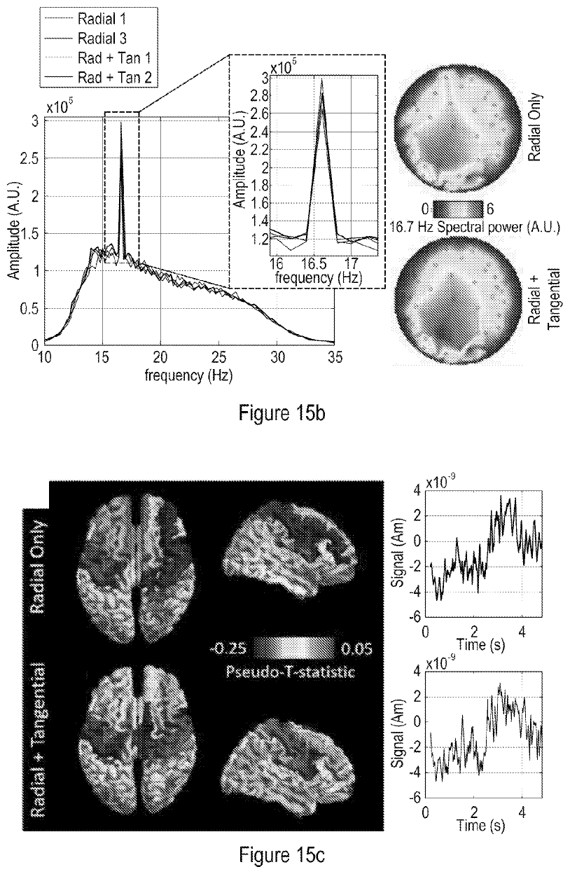

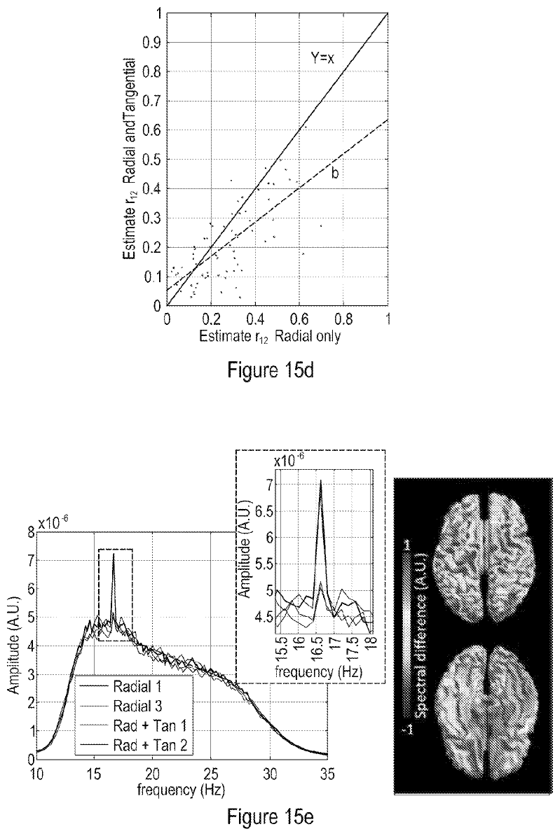

To approximate r₁₂ in experimental data, for each of the 4 runs, the source space topography of the interference pattern (e.g. see FIG. 15b, right hand side) was used as the forward field l₂ of the external interference source S2 and was correlated with the best fitting forward field l₁ for each AAL region according to equation 12. This was done independently for each run and averaged values of r₁₂ from runs 1 and 3 are plotted against averaged values of r₁₂ from runs 2 and 4 in FIG. 15d, discussed further below.

Finally, a conventional analysis was included whereby, at each AAL region, oscillatory amplitude in an active window (1-2 seconds) was contrasted to oscillatory amplitude in a control window (3-4 seconds). This was normalised by the value for the control window to estimate fractional change in beta amplitude induced by the task. The trial averaged beta amplitude for an AAL region in right motor cortex was plotted.

Results: FIG. 15b shows the sensor space data. The line plot (left hand side) shows the average amplitude spectrum obtained from each sensor array 12_5r, 12_5m and for each run (averaged over all trials and sensors, with a clear artifact at ˜16.7 Hz (caused by nearby laboratory equipment). Runs 1 and 3 (using radial sensor array 12_50 are shown in black and blue; runs 1 and 3 (using mixed sensor array 12_5m) are shown in red and green. Note that the artifact is consistent across all 4 runs. The spatial topography/distribution (across sensors in the array) of this artifact for the radial sensor array 12_5r and mixed sensor array 12_5m is shown on the right hand side of FIG. 15b (measured by taking the magnitude of the amplitude spectrum, at this frequency, across all sensors).

The equivalent amplitude spectra for the source space projected data are shown in FIG. 15e. In all 4 runs, the 16.7 Hz artifact has been reduced in relative amplitude compared to the sensor space data in FIG. 15b, however this reduction is significantly more pronounced in runs 2 and 4 using the mixed sensor array 12_5m. The distribution of this improvement across the brain is shown in the inset image to FIG. 15e. These results provide experimental evidence that the primary findings of the theory and simulations presented above can be realised experimentally. The estimated r₁₂ values for each AAL region obtained from both sensor arrays 12_5r, 12_5m are shown in FIG. 15d. If the rotation of sensors in array 12_5m had no effect, then these values would fall along the y=x line (shown in black). However, they consistently fall beneath it (line of best fit shown in blue by line b), implying that r₁₂ is, on average, lowered by sensor rotation in array 12_5m. This indicates that, on average, the forward fields 11 from the sources of interest at AAL regions are less similar and less correlated to the spatial topography of the artifact 12 for the mixed array 12_5m compared to the radial array 12_5r, consistent with above simulation results.

FIG. 15c shows field maps of the change in the reconstructed timecourse (beta modulation, represented by the pseudo-Z-statistic) induced by the task plotted across the different AAL regions for the two arrays 12_5r and 12_5m. The inset to FIG. 15c shows beta amplitude timecourse, averaged over trials, for the two arrays 12_5r and 12_5m. A loss in beta power during movement (the movement related beta decrease—MRBD) and an increase (above baseline) immediately following movement cessation (the post movement beta rebound—PMBR) clearly evident at around 2 seconds. In the field maps, blue indicates a loss of beta power (oscillatory power at the beta frequency band) during the time window where the subject was making controlled left index finger movements. Note that the main effects are well localised to the sensorimotor cortices.

### 4) Discussion

The analytical models and simulations presented in sections 1 and 2 provide unique insights into how a MEG sensor array 12 should be optimised to reduce the error in the reconstructed timecourse and location of the source of interest S1. A first key parameter is Milk the norm of the forward field of the source of interest S1. This quantity can be thought of as the total amount of signal picked up across the sensor array 12. We showed that the total error in a beamformer reconstruction goes as 1/∥l∥, meaning that as ∥l∥ increases, the error will be diminished. The easiest means to increase ∥l∥ is via the addition of extra sensors to an array and so, by effectively tripling the channel count, a bi-axial or a triaxial sensor array immediately adds value to a MEG system 100. A second, more important, key parameter is r₁₂—the forward field correlation between the source of interest S1 and an external source of interference (of no interest) S2. This tells us that if a source of interference S2 has a similar sensor space topography to the source of interest S2, i.e. the measured field pattern looks the same to the sensors, then this will lead to a large error in source reconstruction. However, for a beamformer, the total error on a reconstruction is a non-linear function of r₁₂ meaning that even a modest improvement (reduction) in r₁₂ can yield a relatively large reduction in MEG error. In particular, the introduction of triaxial sensors can have a large effect on r₁₂, and consequently the addition of triaxial sensors, or even rotation of single-axis sensors, enables better source reconstruction in the presence of static of dynamic (e.g. head motion) non-neuromagnetic fields.

Because the array 12 configuration facilitates suppression of interference from sources of non-neuromagnetic fields, the MEG system 100 is able to tolerate higher background fields and greater movement than in conventional MEG systems with radially oriented sensors. The former may relax the shielding requirements of the MSR 40 for the system 100, reducing its cost and complexity, and also allows certain electrical equipment which would ordinarily be located outside the room, such as stimulus equipment, to be located inside the room. This may permit new types of stimulus to be used for MEG measurements. For example, in a conventional MEG system visual stimulus is provided to the subject by projecting images into the MSR 40. The reduced sensitivity to non-neuromagnetic fields afforded by the MEG system 100 of the present invention may allow alternative stimulus equipment, such as virtual reality headsets, to be used. This, coupled with the robustness to movements, may facilitate new developments in MEG and neuroscientific studies.

Although the described embodiments, analysis and results have focused on source reconstruction using a beamformer spatial filter approach, the principle of the method 200, measuring fields in different orientation to reduce the error, applies also to other source reconstruction approaches, including but not limited to dipole fit, or a minimum-norm-estimate algorithms.

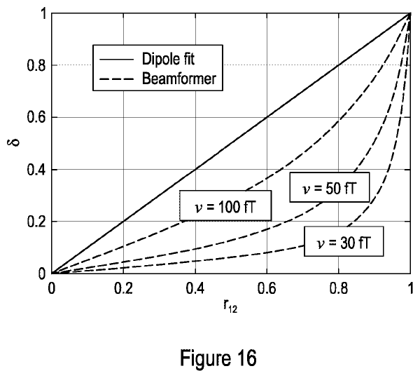

For example, the dipole fit approach is described briefly in appendix C. FIG. 16 shows the dependence of the error 6 associated with non-neuromagnetic fields as a function of r₁₂ for the beamformer and a dipole fit approach for the case where ∥l₁∥=∥l₂∥=1×10⁻¹³ T, source amplitudes are 1 nAm and sensor noise v takes values of 30, 50 and 100 fT. As seen, the reconstruction error b for the dipole fit approach is proportional to r₁₂ (see black line) demonstrating that reducing r₁₂ by measuring different field components in the sensor array 12 will also reduce the error δ for the dipole fit approach. Notably, the error δ is a linear function of r₁₂ for dipole fitting, whereas for the beamformer it is non-linear. The non-linearity in δ(r₁₂) means that a beamformer is better able to suppress interference from non-neuromagnetic fields than the dipole fitting approach, even if the source topographies are highly correlated, and that even a relatively small manipulation of the sensor array design that reduces r₁₂ can result in a potentially large improvement in reconstruction accuracy (reduction in error).

Similarly, although the embodiments described above utilise optically pumped magnetometers (OPMs), it will be appreciated that this is not essential, and other types of sensors 12a-12c with sufficient sensitivity for measuring neuromagnetic fields may be used. For example, although it is preferable from a practical point of view for the sensors 12a-12c to be lightweight and mountable to/in a wearable helmet, which excludes superconducting quantum interference device (SQUID) magnetometers that require cryogenic cooling, this is not essential. As such, in principle, superconducting quantum interference device (SQUID) magnetometers can be used to measure fields in different directions across the array 12 and work the invention. Other emerging quantum sensing technologies, such as nitrogen vacancy magnetometers may also be suitable once the required sensitives for MEG are achieved.

In addition to enabling better differentiation of magnetic field patterns from neural sources within the head and external (to the head) interference (thus improving rejection of signals of no interest), triaxial measurements also offer improved cortical coverage, especially in infants where the brain is positioned proportionally closer to the scalp surface.

By way of example, a single-axis radially-oriented sensor is insensitive to current sources directly beneath it. This is not a problem in conventional MEG (e.g. using SQUIDs) because the sensors are a relatively large distance from the brain, and consequently the radially-oriented field is spatially diffuse, allowing the field from a source to be picked up by single axis radially-oriented sensors that are directly over the source. However, as sensors get closer to the brain, the spatial frequencies of the field become higher, and the gaps between sensors can cause inhomogeneity of spatial sampling (i.e., spatial aliasing).

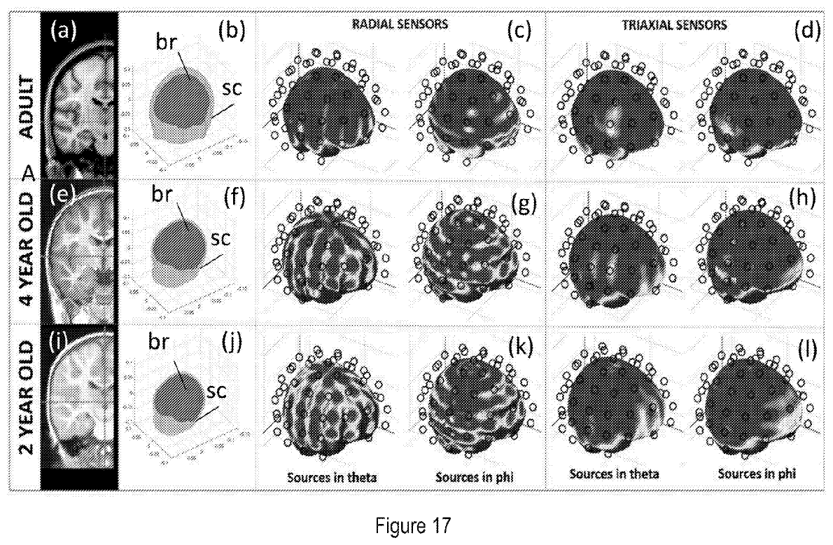

This effect is demonstrated in FIG. 17, which shows the results of simulations of array sensitivity as a function of location in the brain for different aged subjects, discussed below.

Simulations were based on three anatomical models derived from template magnetic resonance images (MRIs) of the brain of an adult, a 4-year-old, and a 2-year-old. These MRIs are shown in FIGS. 17(a), (e) and (i), respectively, and provided an average head geometry for the age group. In each case, a segmentation was applied to derive a surface mesh representing both the scalp and the outer brain. Segmentation was performed using Fieldtrip (see Oostenveld et al. 2011).

FIGS. 17(b), (f) and (j) show a 3D rendering of the resulting head geometry for an adult, a 4-year-old, and a 2-year-old, showing the scalp (sc) and the outer brain (br). As expected, head size grows with age (approximate head circumferences are 58 cm for the adult, 50 cm for the 4-year-old, and 47 cm for the 2-year-old). However, a more dramatic change with age is the proximity of the brain to the scalp surface. Indeed, the average distance from the scalp to the brain is around 15 mm in an adult, but can be as low as 5 mm (in some brain regions) in a 2-year-old. This non-linearity in the development of head geometry is the origin of the MEG sampling problem, discussed above.

Sensor locations around the head were simulated by fitting a sphere to the scalp (sc), and placing 77 equally spaced points on the sphere surface. These locations are then shifted in the radial direction (relative to the sphere) to a point intersecting the scalp (sc), which is taken as the location at which the sensor meets the head. The sensitive volume of the sensor (i.e. where the field measurement is made) was assumed to be 6 mm above the scalp surface (projected radially). Sensors on the underside of the sphere were eliminated to yield a realistic sampling array. In total, 57 sensors were simulated on the adult head, 55 on the 4-year-old, and 57 on the 2-year-old.

Array sensitivity coverage was investigated by simulating shallow dipolar sources located just beneath the brain surface (approximately 5 mm) distributed about the surface of a best fitting sphere. 44,803 dipole locations were simulated for the adult, 43,308 for the 4-year-old and 41,463 for the 2-year-old. For each dipole location, the forward field for dipoles oriented in the polar (theta) and azimuthal (phi) directions was separately computed (i.e. the field that would be measured at the MEG sensors in response to a unit current) using a current dipole model in a single shell volume conductor model. Having computed the field magnitude, bᵢ, at each sensor location/orientation the Frobenius norm of the measured field vector was calculated as fⱼ=\sqrt{Σᵢ₋₁^{N}bᵢ²}, where i indexes MEG channel, N is the total number of channels, and j indexes the source in the brain. The result is an image showing fⱼ as a function of location in the brain, which is referred to as the array sensitivity.

FIGS. 17(c), (g), and (k) show the variation of array sensitivity across the brain of an adult, 4-year old and 2-year old for a radially oriented sensor array. The left-hand images shows sensitivity to dipoles oriented in polar (theta) directions and the right-hand images show the sensitivity to dipoles oriented in azimuthal (phi) directions. The computed values, fⱼ, are normalised by the maximum value to highlight any spatial inhomogeneities in the measured signal, across the brain. Sensor locations are indicated by the open circles.

For an adult, coverage across the brain is approximately uniform, declining with distance from the sensors in areas such as the temporal pole, as expected (see FIG. 17(c)). In contrast, for younger individuals, the simulations in FIGS. 17(g) and (k) show that coverage becomes quite inhomogeneous, with areas of high sensitivity positioned between the sensors, but areas of dramatically lower sensitivity directly beneath the sensors. The spatial signature differs depending on the orientation of the source, as would be expected. This patchy coverage is a direct result of the finite spatial sampling of the sensor array, and the high spatial frequency variation of the magnetic fields measured.

FIGS. 17(d), (h), and (j) show the corresponding variation of array sensitivity across the brain of an adult, 4-year old and 2-year old for a tri-axial sensor array. As can be seen, unlike the radially-oriented array, the triaxial array offers much more uniform coverage than the radially-oriented array, particularly in the 2 and 4-year old results. Whereas a radially oriented sensor is completely insensitive to what is directly beneath it, a tangential measurement is most sensitive to what is beneath it. So, the areas of low sensitivity introduced in the radial array become ‘filled in’ when using a triaxial sensor array. This results in much more uniform coverage. This demonstrates the utility of a triaxial sensor array for imaging electrophysiological phenomena in a child's brain. This will be addressed further in our discussion.

The ability to make high fidelity MEG measurements in infants is an important advantage of triaxial OPM-MEG over conventional scanners, because proximity of sensors to the brain in an infant head can lead to significant sampling problems. The idea that closer sensors are problematic is counter intuitive in MEG, since the closer a sensor gets, the larger the measurable magnetic field, and the more focal its spatial pattern. Thus, closer sensors mean better SNR and spatial resolution. However, the simulations presented in FIG. 17 show that in an infant's head, where distance from the sensor to the brain can be ˜5 mm, the spatial patterns become too focal.

Any practical OPM-MEG system includes a finite number of sensors (currently around 50) and there will always be gaps between sensors. Thus, these highly focal fields become poorly sampled. Consequently, the sensitivity profile varies dramatically across the cortex. This is not an issue in conventional MEG (e.g. using SQUIDs), because the sensors are stepped back from the head to allow for a thermally insulating gap between the scalp and sensors. It is also not an issue for OPM-MEG in adults because the brain is around 15 mm beneath the skull surface (see FIG. 17(a)), and it also is not a problem in EEG because the electric potentials are spatially smeared by the presence of the skull. However, for paediatric OPM-MEG where the brain is very close to the scalp, the simulation results presented here suggest that there is a strong likelihood that effects in the brain could be missed if the region of interest falls within an area of low sensitivity. However, this problem is solved by using triaxial OPM-MEG measurements which provide more uniform coverage.

From reading the present disclosure, other variations and modifications will be apparent to the skilled person. Such variations and modifications may involve equivalent and other features which are already known in the art, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination.

For the sake of completeness it is also stated that the term “comprising” does not exclude other elements or steps, the term “a” or “an” does not exclude a plurality, and any reference signs in the claims shall not be construed as limiting the scope of the claims.

Derivation of the equations in the main text are given in the following appendices.

Appendix A: Analytical Analysis of a Single Source with Gaussian Sensor Noise

Here, we derive an expression for the accuracy of a beamformer reconstruction of a single dipolar source in the brain with Gaussian noise at the sensors. We assume that the location and orientation, 9, chosen for the beamformer coincides with the true location and orientation of the source. We also assume an accurate forward model, meaning that l_{θ}, →l. Via substitution of equation 4 into equation 1, we get

\hat{q}(t)=w^{T}lq(t)+w^{T} [A1]

Here, \hat{q}(t) represents the beamformer estimated reconstruction of the true source timecourse q(t), and w is the N-dimensional vector of beamformer weights tuned to the true source location and orientation. e(t) represents sensor error. By definition (see equation 2) w^{T}l=1, and so

\hat{q}(t)=q(t)+w^{T}e(t) [A2]

and inserting equation 3 we find that

$$\hat{q} ( t ) = q ( t ) + \frac{{L}^{T} {C}^{- 1} e ( t )}{{L}^{T} {C}^{- 1} L} \qquad [ A 3 ]$$

Equation A3 shows that the beamformer estimate is a true reflection of the real source timecourse, q(t), but with additive noise projected through the beamformer weights.

We now consider the analytical form of the data covariance, C and its inverse C⁻¹. For the simple case of a single source, if we assume that the source timecourse is temporally uncorrelated with the sensor noise, we can write

C=E(bb^{T})=E((lq(t)+e(t))(lq(t)+e(t))^{T}) [A4]

where Q represents the standard deviation of q(t). Using the Sherman-Morrison-Woodbury matrix inversion lemma, we can show that

$${C}^{- 1} = \frac{1}{{v}^{2}} ( I - f \frac{{ll}^{T}}{{‖ l ‖}^{2}} ) where \qquad [ A5 ]$$

$$f = \frac{{Q}^{2} {‖ l ‖}^{2}}{{v}^{2} + {Q}^{2} {‖ l ‖}^{2}} \qquad [ A 6 ]$$

is a measure of the effective signal to noise ratio of the source, and scales between 0 and 1. The quantity ∥l∥=\sqrt{l^{T}l} is the Frobenius norm of the forward field vector. We now let

$$P = {w}^{T} Cw = \frac{1}{{l}^{T} {C}^{- 1} l}$$

and substitute equation A5 into equation A3, thus,

$$\hat{q} ( t ) = q ( t ) + P {l}^{T} ( \frac{1}{{v}^{2}} ( I - f \frac{{ll}^{T}}{{‖ l ‖}^{2}} ) ) e ( t ) = q ( t ) + \frac{P}{{v}^{2}} ( {l}^{T} e ( t ) - f \frac{{l}^{T} {ll}^{T} e ( t )}{{‖ l ‖}^{2}} ) . \qquad [ A7 ]$$

Recognising that ∥l∥²=l^{T}l and simplifying we see that

$$\hat{q} ( t ) = q ( t ) + \frac{P}{{v}^{2}} {L}^{T} e ( t ) ( 1 - f ) \qquad [ A8 ]$$

We now also recognise that P depends on the data covariance C and so

$${P}^{- 1} = {l}^{T} {C}^{- 1} l = {l}^{T} ( \frac{1}{{v}^{2}} ( I - f \frac{{ll}^{T}}{{‖ l ‖}^{2}} ) ) l = \frac{1}{{v}^{2}} ( {l}^{T} l - f \frac{{l}^{T} {ll}^{T} l}{{‖ l ‖}^{2}} ) = \frac{{‖ L ‖}^{2}}{{v}^{2}} ( 1 - f ) \qquad [ A9 ]$$

So combining equations A8 and A9 we see that our beamformer estimated timecourse becomes

$$\hat{q} ( t ) = q ( t ) + \frac{1}{{‖ l ‖}^{2}} {l}^{T} e ( t ) . \qquad [ A 10 ]$$

To simplify matters further, we can also write that the vector e(t), which represents the sensor noise across the N sensors can be written as υε(t), where υ is the standard deviation of the sensor noise and we model ε as a gaussian random process with unit standard deviation. So the final expression for \hat{q}(t) becomes

$$\hat{q} ( t ) = q ( t ) + \frac{υ}{{‖ L ‖}^{2}} {L}^{T} ε ( t ) . \qquad [ A 11 ]$$

This equation relates only to a single timepoint, and to compute error over all time, Eₜₒₜ, we calculate the square root of the sum of squared differences between the reconstructed and the true timecourse as per equation 6. Combining equation A11 and equation 6 we get

$${E}_{tot} = \frac{υ}{\sqrt{M} {‖ l ‖}^{2}} \sqrt{{\sum }_{i = 1}^{M} {( {l}^{T} {ε}_{i} )}^{2}} = \frac{υ}{\sqrt{N} {‖ L ‖}^{2}} \sqrt{{\sum }_{i = 1}^{N} {( {\sum }_{j = 1}^{M} {l}_{j} {ε}_{ij} )}^{2}} \qquad [ A12 ]$$

The term (Σⱼ₌₁^{M}lⱼεᵢⱼ)² is simplified considerably because εᵢⱼ is a random process. This means that the cross terms in the square will likely sum to close to zero and can be ignored. Also, noting that E(εᵢⱼ²)=1, this means that

$${E}_{tot} = \frac{υ}{‖ L ‖} \qquad [ A 13 ]$$

In other words, the total error in the beamformer reconstruction, for a single source with random noise, scales linearly with noise amplitude (as one might expect) and is inversely proportional to the Frobenius norm of the forward field from the source.

Appendix B: Analytical Analysis of 2 Sources with Gaussian Noise

Here we extend our analytical treatment to the case of two sources, S1, S2 with Gaussian sensor noise. As shown in the main text, in this case the beamformer reconstruction is given by

[symbol]

=q₁+w₁^{T}l₂q₂+w₁^{T}e=q₁+δq₂+ϵ. [B1]

Note the two error terms. The first (δq₂=w₁^{T}l₂q₂) is interference generated by the second source, and the second (ϵ=w₁^{T}e) is due to projected sensor noise. We now deal with these two error terms separately.

Error from source 2: The magnitude of interference from source S2 is modulated by δ=w₁^{T}l₂. Substituting for the beamformer weights we can write that

$$δ = \frac{{l}_{1}^{T} {C}^{- 1} l}{{l}_{1}^{T} {C}^{- 1} {l}_{1}} = {P}_{1} {l}_{1}^{T} {C}^{- 1} {l}_{2} \qquad [ B 2 ]$$

Where

$${P}_{1} = \frac{1}{{l}_{1}^{T} {C}^{- 1} {l}_{1}}$$

is the projected total power at the location/orientation of the source. To find an expression for the error S, we need an analytical form of both the covariance matrix C and its inverse in the case of two sources S1, S2 with Gaussian noise. Assuming no temporal correlation between either of the two source timecourses, or the sensor noise, then

C=q₁²L₁L₁^{T}+q₂²L₂L₂^{T}+υ²I [B3]

and by the matrix inversion lemma,

$${C}^{- 1} = \frac{1}{{v}^{2}} [ 1 - \frac{1}{1 - {f}_{1} {f}_{2} {cos}^{2} ( {λ}_{12} )} \{ {f}_{1} \frac{{l}_{1} {l}_{1}^{T}}{{‖ {l}_{1} ‖}^{2}} + {f}_{2} \frac{{l}_{2} {l}_{2}^{T}}{{‖ {l}_{2} ‖}^{2}} - {f}_{1} {f}_{2} cos ( {λ}_{12} ) \frac{{l}_{2} {l}_{1}^{T} + {l}_{1} {l}_{2}^{T}}{‖ {l}_{1} ‖ ‖ {l}_{2} ‖} \} ] . \qquad [ B 4 ]$$

As before, f₁ and f₂ represent ratio of signal to sensor noise for the two sources (see equation 13). The quantity

$$cos ( {λ}_{12} ) = \frac{{l}_{1}^{T} {l}_{2}}{‖ {l}_{1} ‖ ‖ {l}_{2} ‖} = {r}_{12} \qquad [ B 5 ]$$

is reflective of the similarity of the forward fields l₁, l₂ for sources S1 and S2; it is mathematically identical to Pearson correlation. Substituting equation B4 into equation B2 we find that

$$δ = \frac{{P}_{1}}{{v}^{2}} [ {l}_{1}^{T} {l}_{2} - \frac{1}{1 - {f}_{1} {f}_{2} {r}_{12}^{2}} \{ {f}_{1} \frac{{l}_{1}^{T} {l}_{1} {l}_{1}^{T} {l}_{2}}{{‖ {l}_{1} ‖}^{2}} + {f}_{2} \frac{{l}_{1}^{T} {l}_{2} {l}_{2}^{T} {l}_{2}}{{‖ {l}_{2} ‖}^{2}} - {f}_{1} {f}_{2} {r}_{12} \frac{{l}_{1}^{T} {l}_{2} {l}_{1}^{T} {l}_{2} + {l}_{1}^{T} {l}_{1} {l}_{2}^{T} {l}_{2}}{‖ {l}_{1} ‖ ‖ {l}_{2} ‖} \} ] \qquad [ B6 ]$$

which simplifies to

$$δ = \frac{{P}_{1} {‖ {l}_{1} ‖}_{F} {‖ {l}_{2} ‖}_{F}}{{v}^{2}} [ \frac{{r}_{12} ( 1 - ( {f}_{1} + {f}_{2} ) + {f}_{1} {f}_{2} )}{1 - {f}_{1} {f}_{2} {r}_{12}} ] \qquad [ B7 ]$$

Noting that

$${P}_{1}^{- 1} = \frac{1}{{v}^{2}} [ {l}_{1}^{T} {l}_{1} - \frac{1}{1 - {f}_{1} {f}_{2} {r}_{12}^{2}} \{ {f}_{1} \frac{{l}_{1}^{T} {l}_{1} {l}_{1}^{T} {l}_{1}}{{‖ {l}_{1} ‖}^{2}} + {f}_{2} \frac{{l}_{1}^{T} {l}_{2} {l}_{z}^{T} {l}_{1}}{{‖ {l}_{2} ‖}^{2}} - {f}_{1} {f}_{2} {r}_{12} \frac{{l}_{1}^{T} {l}_{2} {l}_{1}^{T} {l}_{2} + {l}_{1}^{T} {l}_{1} {l}_{2}^{T} {l}_{2}}{‖ {l}_{1} ‖ ‖ {l}_{2} ‖} \} ] \qquad [ B8 ]$$

which simplifies to

$${P}_{1}^{- 1} = \frac{{‖ {l}_{1} ‖}^{2}}{{v}^{2}} [ \frac{1 - {f}_{1} + ( {f}_{1} {f}_{2} - {f}_{2} ) {r}_{12}^{2}}{( 1 - {f}_{1} {f}_{2} {f}_{12}^{2} )} ] . \qquad [ B9 ]$$

we can now substitute for P₁ in equation B7 giving

$$δ = \frac{‖ {l}_{2} ‖}{‖ {l}_{1} ‖} {r}_{12} [ \frac{1 - {f}_{1} - {f}_{2} + {f}_{1} {f}_{2}}{1 - {f}_{1} + ( {f}_{1} {f}_{2} - {f}_{2} ) {r}_{12}^{2}} ] \qquad [ B10 ]$$

Which simplifies to

$$δ = \frac{‖ {l}_{2} ‖}{‖ {l}_{1} ‖} {r}_{12} [ \frac{1 - {f}_{2}}{1 - {f}_{2} {r}_{12}^{2}} ] \qquad [ B11 ]$$

This expression therefore shows that the extent of interference from source S2 is critically dependent on the parameter r₁₂.

Error from sensor noise: e represents the noise from the sensors projected through the beamformer weights. This is analogous to the sensor noise in the single dipole case (second term in equation A11) but is complicated because the beamformer weights are now based on data from two sources S1, S2. Mathematically, ϵ is given by

ϵ=PL₁^{T}C⁻¹e, [B12]

and substituting for C⁻¹ we get

$$ϵ = \frac{{P}_{1}}{{v}^{2}} [ {l}_{1}^{T} e - \frac{1}{1 - {f}_{1} {f}_{2} {r}_{12}^{2}} \{ {f}_{1} \frac{{l}_{1}^{T} {l}_{1} {l}_{1}^{T} e}{{‖ {l}_{1} ‖}^{2}} + {f}_{2} \frac{{l}_{1}^{T} {l}_{2} {l}_{2}^{T} e}{{‖ {l}_{2} ‖}^{2}} - {f}_{1} {f}_{2} {r}_{12} \frac{{l}_{1}^{T} {l}_{2} {l}_{1}^{T} e + {l}_{1}^{T} {l}_{1} {l}_{2}^{T} e}{‖ {l}_{1} ‖ ‖ {l}_{2} ‖} \} ] , \qquad [ B13 ]$$

which can be written as

$$ϵ = \frac{{P}_{1} ‖ {l}_{1} ‖ \sqrt{N}}{v} [ {r}_{1 e} - \frac{1}{1 - {f}_{1} {f}_{2} {r}_{12}^{2}} \{ {f}_{1} {r}_{1 e} ( 1 - {f}_{2} {r}_{12}^{2} ) + {f}_{2} {r}_{12} {r}_{2 e} ( 1 - {f}_{1} ) \} ] \qquad [ B14 ]$$

Here

$${r}_{1 e} = \frac{{l}_{1}^{T} e}{‖ {l}_{1} ‖ ‖ e ‖}$$

represents the sensor space correlation between the spatial topography of source 1, and the vector representing sensor error. Likewise

$${r}_{2 e} = \frac{{l}_{2}^{T} e}{‖ {l}_{2} ‖ ‖ e ‖}$$

represents the sensor space correlation between the spatial topography of source 2, and the sensor error. Now substituting for P₁ gives

$$ϵ = \frac{v \sqrt{N}}{‖ {l}_{1} ‖} [ {r}_{1 e} ( 1 - \frac{{f}_{1} - {f}_{1} {f}_{2} {r}_{12}^{2}}{1 - {f}_{1} {f}_{2} {r}_{12}^{2}} ) - {r}_{2 e} \frac{( {f}_{2} {r}_{12} - {f}_{1} {f}_{2} {r}_{12} )}{1 - {f}_{1} {f}_{2} {r}_{12}^{2}} ] [ \frac{1 - {f}_{1} {f}_{2} {r}_{12}^{2}}{1 - {f}_{1} + ( {f}_{1} {f}_{2} - {f}_{2} ) {r}_{12}^{2}} ] \qquad [ B15 ]$$

Which simplifies to give

$$ϵ = \frac{v \sqrt{N}}{‖ {l}_{1} ‖} [ {r}_{1 e} ( \frac{1}{1 - {f}_{2} {r}_{12}^{2}} ) - {r}_{2 e} ( \frac{{f}_{2} {r}_{12}}{1 - {f}_{2} {r}_{12}^{2}} ) ] \qquad [ B16 ]$$

Error over all time: q₂ is a function of time. For this reason, it is useful to consider the total error across all timepoints. Substituting equation B1 into equation 15 we find that

$${E}_{Tot}^{2} = \frac{1}{M} {\sum }_{i = 1}^{M} {( δ {q}_{2 i} + {ϵ}_{i} )}^{2} , \qquad [ B17 ]$$

where i indexes time. Assuming that q₂ᵢ and ϵᵢ are temporally uncorrelated, we can write the total error as two independent terms, thus

$${E}_{2 sour}^{2} = \frac{1}{M} {\sum }_{i = 1}^{M} [ {( δ {q}_{2 i} )}^{2} + {( {ϵ}_{i} )}^{2} ] . \qquad [ B18 ]$$

Noting that E(q₂ᵢ)²=Q₂² the total error due to interference from the second source is given by

$${E}_{s o u r c e}^{2} = \frac{1}{M} {\sum }_{i = 1}^{M} {( δ {q}_{2 i} )}^{2} = {δ}^{2} {Q}_{2}^{2} = \frac{{Q}_{2}^{2} {‖ {l}_{2} ‖}^{2}}{{‖ {l}_{1} ‖}^{2}} {{r}_{12}^{2} [ \frac{1 - {f}_{2}}{1 - {f}_{2} {r}_{12}^{2}} ]}^{2} . \qquad [ B19 ]$$

Error due to sensor noise is somewhat more complex to deal with, but from equation B16 we can write that

$${E}_{n o i s e}^{2} = \frac{1}{M} {\sum }_{i = 1}^{M} {( {ϵ}_{i} )}^{2} = \frac{1}{M} {\sum }_{i = 1}^{M} \frac{{v}^{2} N}{{‖ {l}_{1} ‖}^{2}} {( a {r}_{1 e} - b {r}_{2 e} )}^{2} \qquad [ B20 ]$$

Where

$$a = ( \frac{1}{1 - {f}_{2} {r}_{12}^{2}} ) and b = ( \frac{{f}_{2} {r}_{12}}{1 - {f}_{2} {r}_{12}^{2}} ) = {f}_{2} {r}_{12} a .$$

Expanding the square this becomes

$${E}_{noise}^{2} = \frac{1}{M} {\sum }_{i = 1}^{M} \frac{{v}^{2} N}{{‖ {l}_{1} ‖}^{2}} ( {a}^{2} {r}_{1 e}^{2} + {b}^{2} {r}_{2 e}^{2} - 2 a b {r}_{1 e} {r}_{2 e} ) \qquad [ B21 ]$$

To simplify this, we first note that

$${r}_{1 e} {r}_{2 e} = \frac{{l}_{1}^{T} e}{‖ {l}_{1} ‖ ‖ e ‖} \frac{{l}_{2}^{T} e}{‖ {l}_{2} ‖ ‖ e ‖} = \frac{{l}_{1}^{T} e {e}^{T} {l}_{2}}{‖ {l}_{1} ‖ ‖ {l}_{2} ‖ {‖ e ‖}^{2}} , \qquad [ B22 ]$$

And because e is a Gaussian random process, when summed over many iterations E(ee^{T})=υ²I. ∥e∥, the Frobenious norm of the error is simply given by E(∥e∥)=\sqrt{Nυ²} where N is the total number of MEG channels. Consequently we can write that

$$E ( {r}_{1 e} {r}_{2 e} ) = \frac{{l}_{1}^{T} {l}_{2}}{‖ {l}_{1} ‖ ‖ {l}_{2} ‖ N} = \frac{1}{N} {r}_{12} \qquad [ B23 ]$$

Next, we examine r₁ₑ² and note that

$${r}_{1 e}^{2} = \frac{{( {l}_{1}^{T} e )}^{2}}{{‖ {l}_{1} ‖}^{2} {‖ e ‖}^{2}} \qquad [ B24 ]$$

Again we take advantage of the fact that e is a Gaussian random process, so we can write (l₁^{T}e)²=(Σₛ₌₁^{N}l₁ₛeₛ)²≈Σₛ₌₁^{N}(l₁ₛeₛ)² because on average the cross terms in the square will sum to zero. Because E(eₛ²)=υ² then Σₛ₌₁^{N}(l₁ₛeₛ)²=υ² then Σₛ₌₁^{N}(l₁ₛeₛ)²=υ²Σₛ₌₁^{N}(l₁ₛ)²υ²∥l₁∥². Further, since E(∥e∥²)=Nυ² we find that

$$E ( {r}_{1 e}^{2} ) = \frac{1}{N} . \qquad [ B25 ]$$

The same argument can be made to show that

$$E ( {r}_{2 e}^{2} ) = \frac{1}{N} . \qquad [ B26 ]$$

Substituting Equations B25, B26 and B23 into B21 we see that

$${E}_{noise}^{2} = \frac{1}{M} \frac{{v}^{2} N}{{‖ {l}_{1} ‖}^{2}} ( M {a}^{2} \frac{1}{N} + M {b}^{2} \frac{1}{N} - M 2 a b \frac{1}{N} {r}_{12} ) \qquad [ B27 ]$$

Substituting for a and b this then simplifies to give

$${E}_{noise}^{2} = \frac{{v}^{2}}{{‖ {l}_{1} ‖}^{2}} ( \frac{1 + {f}_{2}^{2} {r}_{12}^{2} - 2 {f}_{2} {r}_{12}^{2}}{{( 1 - {f}_{2} {r}_{12}^{2} )}^{2}} ) \qquad [ B28 ]$$

### Appendix C: Analytical Analysis for a Dipole Fit Approach

For most source reconstruction algorithms, the reconstructed source signal can be written as a weighted sum of sensor measurements, as in equation 1. For a dipole fit, the weights are given by

$${w}_{d θ}^{T} = \frac{{l}_{d θ}^{T}}{{l}_{d θ}^{T} {l}_{d θ}} \qquad [ C1 ]$$

i.e. they are a scaled version of the lead field and do not depend on the data covariance—as in a beamformer. To understand how this affects reconstruction error, we can apply these weights to the analytical formulation of the sensor data for the case of two sources with random noise (i.e. combine equation 8 and equation C1). Assuming an accurate lead field (l_{dθ}→l₁),

$$= \frac{{l}_{1}^{T}}{{l}_{1}^{T} {l}_{1}} {l}_{1} {q}_{1} + \frac{{l}_{1}^{T}}{{1}_{1}^{T} {l}_{1}} {l}_{2} {q}_{2} + \frac{{l}_{1}^{T}}{{1}_{1}^{T} {l}_{1}} e \qquad [ C2 ]$$

Substituting

$${r}_{12} = \frac{{l}_{1}^{T} {l}_{2}}{‖ {l}_{1} ‖ ‖ {l}_{2} ‖} ,$$

we see that

$$= {q}_{1} + \frac{‖ {l}_{2} ‖}{‖ {l}_{1} ‖} {r}_{12} {q}_{2} + \frac{{l}_{1}^{l} e}{{‖ {l}_{1} ‖}^{2}} \qquad [ C3 ]$$

Or alternatively

$$= {q}_{1} + \frac{‖ {l}_{2} ‖}{‖ {l}_{1} ‖} {r}_{12} {q}_{2} + \frac{‖ e ‖}{‖ {l}_{1} ‖} {r}_{1 e} \qquad [ C3 ]$$

Note that the error generated by the contribution of source S2 to the reconstruction is a linear function of r₁₂, whereas for the beamformer, the equivalent term (δ, equation B11) is non-linear. These two functions are shown plotted in FIG. 16.

The invention claimed is:

1. A method of reducing error in magnetoencephalography arising from the presence of a non-neuromagnetic field, comprising:
measuring, using a sensor array for measuring neuromagnetic fields, magnetic field at a plurality of discrete locations around a subject's head to provide sensor data, wherein the magnetic field measured at at least some of the locations includes a neuromagnetic field from a source of interest within a subject's brain and a non-neuromagnetic field from a source of no interest external to the brain, comprising:
measuring, at at least a first subset of the locations, a magnetic field along a first direction relative to a radial axis intersecting the respective location, and
measuring, at at least a second subset of the locations, a magnetic field along a second direction relative to a radial axis intersecting the respective location which is different to the first direction; and
performing source reconstruction using the sensor data.

2. The method of claim 1, wherein the error associated with the non-neuromagnetic field includes an error in the reconstructed timecourse and/or location of a source of interest within the subject's brain.

3. The method of claim 1, wherein the non-neuromagnetic field includes a substantially spatially uniform background magnetic field and/or a spatially non-uniform background magnetic field; and/or, wherein the non-neuromagnetic field includes a static background magnetic field and/or a dynamic background magnetic field; and optionally or preferably, wherein the dynamic background magnetic field is a result of relative movement of the sensor array and a static non-neuromagnetic field.

4. The method of claim 1, comprising measuring, at each location or at least some locations, a magnetic field along the first and second directions.

5. The method of claim 1, wherein the first direction and the second direction are substantially orthogonal; and/or wherein the first direction and the second direction are substantially the same at each location.

6. The method of claim 1, further comprising and measuring, at at least a third subset of the locations, a magnetic field along a third direction relative to a radial axis intersecting the respective location which is different to the first direction and the second direction.

7. The method of claim 6, comprising measuring, at each location or at least some locations, a magnetic field along the first, second and third directions.

8. The method of claim 6, wherein the third direction is substantially orthogonal to the first direction and/or the second direction; and/or wherein the third direction is substantially the same at each location.

9. The method of claim 1, wherein the second direction is aligned substantially parallel to the radial axis at the respective location.

10. The method of claim 1, wherein performing source reconstruction comprising using a beamformer or a dipole fit or a minimum-norm estimate approach.

11. The method of claim 1, where each sensor is or comprises an optically pumped magnetometer.

12. Use of a sensor array for measuring neuromagnetic fields at a plurality of discrete locations around a subject's head for reducing error in magnetoencephalography associated with non-neuromagnetic fields, wherein:
at least a first subset of sensors are configured to measure a magnetic field along a first direction relative to a radial axis intersecting the respective sensor location; and
at least a second subset of sensors are configured to measure a magnetic field along a second direction relative to a radial axis intersecting the respective sensor location that is different to the first direction.

13. Use of the sensor array according to claim 12, wherein the error associated with the non-neuromagnetic field includes an error in the reconstructed timecourse and/or location of a source of interest within the subject's brain.

14. Use of the sensor array according to claim 12, wherein the non-neuromagnetic field includes a substantially spatially uniform background magnetic field and/or a spatially non-uniform background magnetic field; and/or wherein the non-neuromagnetic field includes a static background magnetic field and/or a dynamic background magnetic field; and optionally or preferably, wherein the dynamic background magnetic field is a result of relative movement of the sensor array and the non-neuromagnetic field.

15. Use of the sensor array according to claim 12, wherein all of the sensors or at least some of the sensors are dual-axis sensors configured to measure a magnetic field along the first direction and the second direction.

16. Use of the sensor array according to claim 12, wherein the first direction and the second direction are substantially orthogonal; and/or wherein the first direction and the second direction are substantially the same at each sensor location.

17. Use of the sensor array according to claim 12, wherein at least a third subset of sensors are configured to measure a magnetic field along a third direction relative to a radial axis intersecting the respective sensor location which is different to the first direction and the second direction.

18. Use of the sensor array according to claim 17, wherein all of the sensors or at least some of the sensors are tri-axial sensors configured to measure a magnetic field along the first, second and third directions.

19. Use of the sensor array according to claim 17, wherein the third direction is substantially orthogonal to the first direction and/or the second direction; and/or wherein the third direction is substantially the same at each sensor location.

20. Use of the sensor array according to any of claims 12 to 19 claim 12, wherein the second direction is aligned substantially parallel to the radial axis at the respective sensor location.

21. Use of the sensor array according to any of claims 12 to 20 claim 12, where each sensor is or comprises an optically pumped magnetometer.

22. A system for magnetoencephalography, comprising:
a sensor array for measuring neuromagnetic fields at a plurality of discrete locations around a subject's head and output sensor data, wherein:
at least a first subset of sensors are configured to measure a magnetic field along a first direction relative to a radial axis intersecting the respective sensor location; and
at least a second subset of sensors are configured to measure a magnetic field along a second direction relative to a radial axis intersecting the respective sensor location that is different to the first direction; and
a processing module configured to perform source reconstruction using the sensor data,
wherein the sensor data comprises at least one magnetic field measured at each sensor location, and at least some of the measured magnetic fields include a neuromagnetic field from a source of interest within a subject's brain and a non-neuromagnetic field from a source of no interest external to the brain, and
wherein the system is configured to reduce error in magnetoencephalography associated with the non-neuromagnetic field.

23. The system of claim 22, wherein each sensor of the array comprises an optically pumped magnetometer; and/or wherein the processing module is configured to perform source reconstruction using a beamformer, dipole fit, or a minimum-norm-estimate algorithm.

24. The system of claim 23, further comprising a wearable helmet comprising the sensor array; and optionally wherein the helmet is substantially rigid or flexible.

25. The system of claim 22, wherein all of the sensors or at least some of the sensors are tri-axial sensors configured to measure a magnetic field along the first, second and third directions; and optionally wherein:
the third direction is substantially orthogonal to the first direction and/or the second direction and/or
the second direction is aligned substantially parallel to the radial axis at the respective sensor location.

26. (canceled)

27. A method of performing magnetoencephalography on a child, comprising:
measuring, using an array of optically pumped magnetometers, magnetic field along three orthogonal directions at a plurality of discrete locations around the child's head to provide triaxial sensor data; and
performing source reconstruction using the triaxial sensor data,

28. A method of improving spatial sensitivity coverage in magnetoencephalography performed on a child using an array of optically pumped magnetometers, comprising:
measuring, using the array of optically pumped magnetometers, magnetic field along three orthogonal directions at a plurality of discrete locations around the child's head to provide triaxial sensor data; and
performing source reconstruction using the triaxial sensor data,

29. The method according to claim 27, wherein the child has an age of less than 5 years old.

30. Use of a triaxial sensor array in magnetoencephalography performed on a child for improved array sensitivity coverage, wherein each triaxial sensor comprises an optically pumped magnetometer configured to measure magnetic field along three orthogonal axes.

31. The use of the triaxial sensor array according to claim 30, wherein the child has an age of less than 5 years old.
